(19) United States
(12) Patent Application Publication
(10) Pub. No.: US 2017/0121752 A1
(43) Pub. Date: May 4, 2017

(54) DETECTION OF ACRYLIC ACID

(71) Applicant: AGENCY FOR SCIENCE, TECHNOLOGY AND RESEARCH, Singapore (SG)

(72) Inventors: Yin Nah TEO, Singapore (SG); Zhi Yi LEE, Singapore (SG); Farid GHADESSY, Singapore (SG); Sarada Srinivasa RAGHAVAN, Singapore (SG)

(21) Appl. No.: 15/311,185

(22) PCT Filed: May 14, 2015
(86) PCT No.: PCT/SG2015/050116

§ 371 Date: Nov. 14, 2016

(30) Foreign Application Priority Data
May 14, 2014 (SG) 10201402318X

(51) Int. Cl. C12Q 1/02; C09K 11/06; G01N 21/64

(52) U.S. Cl. CPC C12Q 1/02; G01N 21/6428; C09K 11/06; G01N 2021/6432; G01N 2021/7786

(57) ABSTRACT

There is provided a method for detecting the presence or absence of acrylic acid or its derivatives thereof in a sample, the method comprising the steps of: (a) introducing a probe comprising a diaryltetrazole compound to the sample; (b) exposing said sample to light; and (c) detecting the presence or absence of acrylic acid or its derivatives thereof in the sample based on fluorescence emitted by the sample after step (c).

### TECHNICAL FIELD

The present invention generally relates to a rapid and sensitive method for detecting the presence or absence of acrylic acid or its derivatives thereof. The present invention also relates to a probe for detecting the presence or absence of acrylic acid or its derivatives thereof.

### BACKGROUND ART

Acrylic acid can be widely used as a feedstock for the industrial production of a wide range of acrylate esters and polymers for applications such as plastics, latex, superabsorbent polymers, surface coatings, textiles, adhesives and sealants. The global demand for acrylic acid was more than USD $13.6 billion in 2012 and may increase to USD $20.0 billion by 2018.

One of the commonly used raw materials in the production of acrylic acid may be propylene, which can typically be derived from petrochemical sources. However, in recent years, there appears to be great interest in producing acrylic acid through alternative, sustainable, biorenewable sources. In order to produce green acrylic acid, including its derivatives thereof, or those derived from biomass and waste products, the ability to detect acrylic acid or its derivatives thereof with a sensitive and specific assay is needed.

A rapid, high-throughput detection method for acrylic acid or its derivatives may be used to facilitate both strain engineering of microbial acrylic acid producers and engineering of relevant enzymes for improved acrylic acid production in vivo. A highly sensitive method may also be required to enhance the accuracy of determining the presence or absence of any acrylic acid or its derivatives. Such methods may also be used for detecting acrylate contaminants from plastics. This highly sensitive method may be used to detect environmental contamination caused by acrylate contaminants in rivers or drinking water.

Some currently available methods for detecting acrylic acid or its derivatives may comprise chromatographic methods such as gas chromatography (GC) and high pressure liquid chromatography (HPLC) coupled with mass spectrometry detection. However, the limitations present in these chromatographic methods may include tedious sample preparation procedures and chemical derivatization of the compounds to be used as sensors for detection. These methods tend to suffer from low throughputs and are likely to be unsuitable for screening large quantities of chemicals.

Accordingly, there is a need to provide a method for detecting the presence or absence of acrylic acid or its derivatives thereof that overcomes, or at least ameliorates, one or more of the disadvantages described above. Some of these advantages may include improved sensitivity and throughput for detection. This method should also be capable of providing a more rapid detection of the presence or absence of acrylic acid or its derivatives thereof.

There is a further need to provide a probe that is capable of rapidly detecting the presence or absence of acrylic acid or its derivatives thereof. This probe should also be capable of overcoming, or at least ameliorating, one or more of the disadvantages described above.

### SUMMARY OF INVENTION

According to a first aspect, there is provided a method for detecting the presence or absence of acrylic acid or its derivatives thereof in a sample, the method comprising the steps of:

(a) introducing a probe comprising a diaryltetrazole compound to the sample;
(b) exposing said sample to light; and
(c) detecting the presence or absence of acrylic acid or its derivatives thereof in the sample based on fluorescence emitted by the sample after said step (c).

Compared to conventional methods of detection, for instance, gas or liquid chromatography, the method as described above allows for rapid and high throughput sensing. The need for tedious sample extraction/preparation or chemical derivatization of the compounds to be used as detection sensors may be advantageously eliminated. The use of bulky detection apparatus may also be mitigated. The acrylic acid or it derivatives capable of being detected by this method may comprise, but not limited to, acrylamide, acrylate esters or other acrylate based compounds.

Advantageously, the present method which relies on the reaction between a diaryltetrazole and an acrylic acid or its derivatives thereof, may be capable of being completed within 90 seconds upon photoactivation, thereby improving the speed of detection. The light used for photoactivation may be ultraviolet light (UV).

The diaryltetrazole compound as described in the above method may have the formula (I):

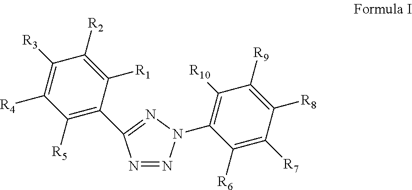

wherein each of R₁ to R₁₀ is independently selected from the group consisting of hydrogen, oxygen, sulfur, halogen, hydroxyl, optionally substituted alkyl, optionally substituted acyl, optionally substituted ester, optionally substituted amino, optionally substituted amine, optionally substituted amide, optionally substituted carboxylic acid, optionally substituted carbonyl, optionally substituted urea, optionally substituted alkoxy, optionally substituted alkyloxy, optionally substituted alkenyl, optionally substituted alkynyl, optionally substituted sulfonamide, optionally substituted aminosulfonamide, optionally substituted sulfonylurea, optionally substituted oxime, optionally substituted cycloalkyl, optionally substituted aryl, optionally substituted heterocycloalkyl and optionally substituted heteroaryl. Advantageously, the diaryltetrazole may independently comprise different types of optional substituents at each of R₁ to R₁₀ for improving the efficiency or accuracy of the detection.

Particularly, the diaryltetrazole compound may be selected from the group consisting of:

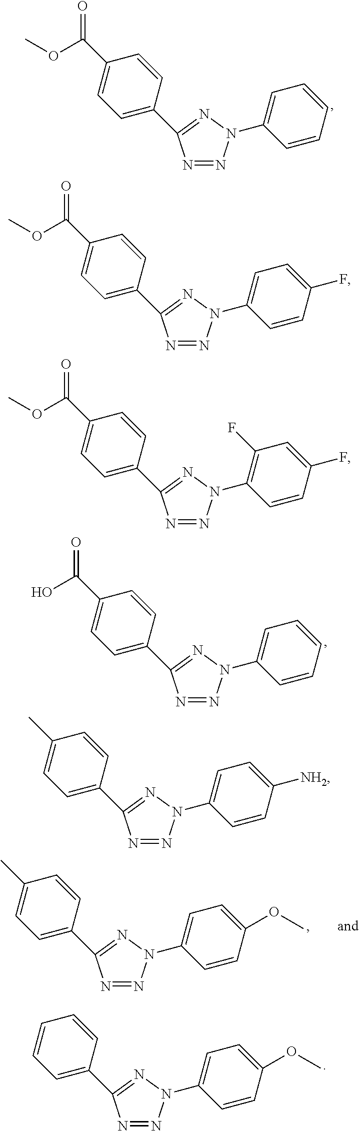

The diaryltetrazole compound introduced in step (a) of the present method may be present at a concentration of at least 1 nM in the sample.

In the method as described above, the probe may be biotinylated. By attaching biotinylation reagents to the probe, substances such as streptavidin and avidin with an extremely high affinity, fast on-rate, and high specificity may be used to isolate the biotinylated probes. Biotinylation enhances the accuracy of detection as it allows the biotinylated probe to be captured more efficiently. Furthermore, affinity purification of biotinylated probe-acrylic acid/acrylic acid derivative conjugates potentially removes substances present in complex (e.g. biological) samples that may confound analysis, for example by autofluorescence.

In step (b) of the present method disclosed, the exposure may occur at any wavelength in the range of 10 nm to 1 mm. Particularly, the wavelength may be 302 nm. Detection using these wavelengths advantageously avoids the use of complex light-emitting sources. The exposure of the sample to light may occur under acidic or alkaline conditions.

In step (b) of the method as described above, the step of exposure may further comprise the step of forming a reactive intermediate. This reactive intermediate may be a compound comprising a nitrile imine dipole. This nitrile imine dipole is capable of reacting with acrylic acid or its derivatives thereof to produce a pyrazoline cycloadduct that may be fluorescent. Advantageously, these steps allow acrylic acid or its derivatives to be detected via a fluorimetric method. Accordingly, the fluorescent sample obtained after exposure to light may be a cycloadduct comprising a fluorescent pyrazoline.

To enhance accuracy of detection, the acrylic acid or its derivatives thereof may have to be present at a concentration of at least 100 nM before introducing the probe to the sample containing the acrylic acid or its derivatives thereof.

The present method may be used to detect the presence or absence of acrylic acid or its derivatives thereof in microorganisms without inducing cytotoxicity in these organisms, for instance, in bacterium.

According to another aspect, there is provided a probe for detecting the presence or absence of acrylic acid or its derivatives thereof in a sample, wherein the probe comprises a diaryltetrazole compound. This probe may provide the advantages as described above. The diaryltetrazole compound may have the formula (I) as indicated above.

The probe as defined above may be selected from the group consisting of:

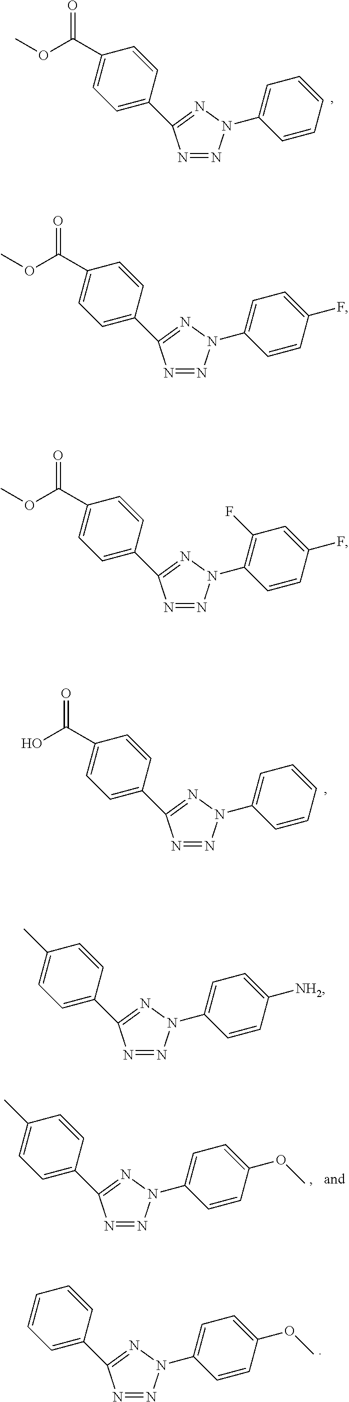

Advantageously, the probe may be biotinylated as described above.

According to another aspect, there is provided the use of a probe as defined above for the detection of the presence or absence of acrylic acid or its derivatives. The use of this probe in this manner provides the advantages as described above.

According to another aspect, there is provided a kit comprising the probe as defined above for detecting the presence or absence of acrylic acid or its derivatives, wherein the probe is contacted with the acrylic acid or its derivatives. This kit is capable of providing the advantages as described above.

### DEFINITIONS

The following words and terms used herein shall have the meaning indicated:

In the definitions of a number of substituents below or as described throughout this disclosure, the substituent groups may be a terminal group or a bridging group. This is intended to signify that the use of the temi is intended to encompass the situation where the group is a linker between two other portions of the molecule as well as where it is a terminal moiety. Using the term alkyl as an example, some publications would use the term “alkylene” for a bridging group and hence in these other publications there is a distinction between the terms “alkyl” (terminal group) and “alkylene” (bridging group). In the present disclosure, no such distinction is made and most groups may be either a bridging group or a terminal group.

The phrase “optionally substituted” is to be interpreted broadly to mean that the group to which this term refers to may be unsubstituted, or may be substituted with one or more groups independently selected from, but not limited to, oxygen, sulfur, halogen, alkyl, acyl, ester, amino, amide, carboxylic acid, carbonyl, urea, alkoxy, alkyloxy, alkenyl, alkynyl, sulfonamide, aminosulfonamide, sulfonylurea, oxime, cycloalkyl, aryl, heterocycloalkyl and heteroaryl. Usually these groups have 1 to 12 carbon atoms, if they contain carbon atoms.

The term “halogen” or variants such as “halide” or “halo” as used herein refers to fluorine, chlorine, bromine and iodine or a group 17 element of the periodic table.

The term “alkyl” may refer to a straight- or branched-chain alkyl group having from 1 to 12 carbon atoms or any number of carbon atoms falling within this range in the chain. Exemplary alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and the like.

The term “acyl” may mean a —C(O)—R radical, wherein R is an optionally substituted C₁-C₁₂-alkyl, C₂-C₁₂-alkenyl, cycloalkyl having 3 to 12 carbon atoms, or aryl having 6 or more carbon atoms, or a 5 to 6 ring membered heterocycloalkyl or heteroaryl group having 1 to 3 hetero atoms select from N, S or O.

The term “ester” includes within its meaning —O—C(O)-alkyl- and —C(O)—O-alkyl- groups.

The term “amino” as used herein may refer to groups of the form —NR^{a}R^{b} wherein R^{a} and R^{b} are individually selected from the group including but not limited to hydrogen, optionally substituted alkyl, optionally substituted alkenyl, optionally substituted alkynyl, and optionally substituted aryl groups. The term “amino” may include an amine group (i.e. —NH₂) or a substituted amine group as defined below.

The term “amide” as used herein may refer to groups of the form —C(O)NR^{c}-alkyl- wherein R^{c} is selected from the group including but not limited to hydrogen, optionally substituted alkyl, optionally substituted alkenyl, and optionally substituted aryl groups.

The term “amine” as used herein refers to groups of the form NR^{d}R^{e}-alkyl- wherein R^{d} and R^{e} are individually selected from the group including but not limited to hydrogen, optionally substituted alkyl, optionally substituted alkenyl, and optionally substituted aryl groups. The -alkyl- groups in the “amide” and “amine” can be optionally substituted and preferably have 2 to 12 carbon atoms, more preferably 2 to 6 carbon atoms or any number of carbon atoms falling within these ranges.

The term “carboxylic acid” or variants such as “carboxyl” may be intended to refer to a molecule having the group having —C(O)OH.

The term “carbonyl” may refer to a molecule having the group R^{f}—C(O)—R^{g}, wherein R^{f} and R^{g} may be an optionally substituted C₁-C₁₂-alkyl, C₂-C₁₂-alkenyl, cycloalkyl having 3 to 12 carbon atoms, or aryl having 6 or more carbon atoms, or a 5 to 6 ring membered heterocycloalkyl or heteroaryl group having 1 to 3 hetero atoms select from N, S or O. This term may encompass a ketone.

The term “alkoxy” or variants such as “alkoxide” or “alkyloxy” as used herein may refer to an O-alkyl radical. Representative examples include, for example, methoxy, ethoxy, n-propoxy, isopropoxy, tert-butoxy, and the like.

The term “alkenyl group” includes within its meaning divalent (“alkenylene”) straight or branched chain unsaturated aliphatic hydrocarbon groups having from 2 to 12 carbon atoms or any number of carbon atoms falling within this range and having at least one double bond, of either E, Z, cis or trans stereochemistry where applicable, anywhere in the alkyl chain. Examples of alkenyl groups include but are not limited to ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 2-heptentyl, 3-heptenyl, 1-octenyl, 1-nonenyl, 1-decenyl, and the like.

The term “alkynyl” as used herein, unless otherwise specified, may refer to a branched or unbranched hydrocarbon group of 2 to 12 or any number of carbon atoms falling within this range and containing at least one triple bond, such as acetylenyl, ethynyl, n-propynyl, isopropynyl, n-butynyl, isobutynyl, t-butynyl, octynyl, decynyl and the like.

The term “cycloalkyl” as used herein may refer to a stable non-aromatic monocyclic or polycyclic hydrocarbon radical consisting solely of carbon and hydrogen atoms, which may include fused or bridged ring systems, having from three to fifteen carbon atoms or any number of carbon atoms falling within this range. The “cycloalkyl” may be attached to the rest of the molecule by a single bond. The “cycloalkyl” may be saturated i.e. containing single C—C bonds only. Examples of monocyclic cycloalkyls include, e.g., cyclopropyl, cyclobutyl, cyclopentyl, cyclohexyl, cycloheptyl, and cyclooctyl.

The term “aromatic group”, or variants such as “aryl” or “arylene” as used herein may refer to monovalent (“aryl”) and divalent (“arylene”) single, polynuclear, conjugated and fused residues of aromatic hydrocarbons having from 6 to 12 carbon atoms or any number of carbon atoms falling within this range. Examples of such groups include phenyl, biphenyl, naphthyl, phenanthrenyl, and the like.

The term “heterocycloalkyl” may refer to a saturated monocyclic, bicyclic, or polycyclic ring containing at least one heteroatom selected from nitrogen, sulfur, oxygen, preferably from 1 to 3 heteroatoms in at least one ring. Each ring may be from 3 to 12 membered or having any number of carbon atoms within this range. Examples of heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane, and 1,4 oxathiapane.

The term “heteroalkyl” refers to a straight- or branched-chain alkyl group having from 2 to 12 atoms in the chain or any number of atoms falling within this range, one or more of which is a heteroatom selected from S, O and N. Exemplary heteroalkyls include alkyl ethers, secondary and tertiary alkyl amines, alkyl sulfides, and the like.

The term “heteroaryl” as used herein may refer to an aromatic monocyclic or multicyclic ring system comprising about 5 to about 12 ring atoms, preferably about 5 to about 10 ring atoms or any number of atoms falling within this range, in which one or more of the ring atoms is an element other than carbon, for example nitrogen, oxygen or sulfur, alone or in combination. The term “heteroaryl” may also include a heteroaryl as defined above fused to an aryl as defined above. Non-limiting examples of suitable hetcroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like. Heteroaryl groups may be optionally substituted.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF EMBODIMENTS

Exemplary, non-limiting embodiments of the method as described above will now be disclosed.

The method for detecting the presence or absence of acrylic acid or its derivatives thereof in a sample may comprise the steps of: (a) introducing a probe comprising a diaryltetrazole compound to the sample; (b) exposing the sample to light; and (c), detecting the presence or absence of acrylic acid or its derivatives thereof in the sample based on fluorescence emitted by the sample after step (c).

Advantageously, the method may provide rapid detection of acrylic acid or its derivatives with high throughput as compared to conventional methods such as, but not limited to GC, gas chromatography mass spectroscopy (GCMS), liquid chromatography or HPLC etc. The method may utilize a probe that is non-toxic and hence allows the present method to be used for detecting acrylic acid or its derivatives in vitro or in vivo.

The acrylic acid or its derivatives thereof that may be detected by the present method may comprise, but not limited to, acrylamide, acrylate esters or any other acrylate based compounds. These acrylates derivatives may also be any acrylate salts, esters, conjugate bases of acrylic acid or its derivatives. Basically these derivatives or acrylates may contain vinyl groups, that is, two carbon atoms double bonded to each other, which is in turn directly attached to the carbonyl carbon. Acrylates or acrylate based compounds may also encompass acrylate based polymers or methacrylates (the salts and esters of methacrylic acid).

The sample as described above may be any sample containing acrylic acid or its derivatives thereof. The sample may comprise a microorganism. This organism may be a virus, a bacterium, any animal or plant cell etc. This organism may be capable of producing any acrylic acid or its derivative thereof. Examples of acrylic acid producing bacterium may comprise Clostridium propionicum and Megasphaera elsdenii.

In step (a) of the present method, a probe may be introduced into the targeted sample. The probe may comprise a diaryltetrazole compound. The diaryltetrazole compound may have the formula

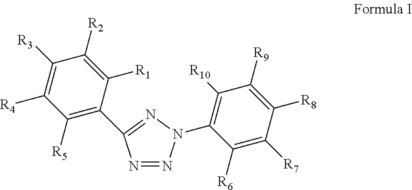

wherein each of R₁ to R₁₀ is independently selected from the group consisting of hydrogen, oxygen, sulfur, hydroxyl, halogen, optionally substituted alkyl, optionally substituted acyl, optionally substituted ester, optionally substituted amino, optionally substituted amine, optionally substituted amide, optionally substituted carboxylic acid, optionally substituted carbonyl, optionally substituted urea, optionally substituted alkoxy, optionally substituted alkyloxy, optionally substituted alkenyl, optionally substituted alkynyl, optionally substituted sulfonamide, optionally substituted aminosulfonamide, optionally substituted sulfonylurea, optionally substituted oxime, optionally substituted cycloalkyl, optionally substituted aryl, optionally substituted heterocycloalkyl, optionally substituted heteroalkyl, optionally substituted alcohol and optionally substituted heteroaryl.

Some examples of diaryltetrazole compounds encompass by formula (I) may be selected from the group consisting of:

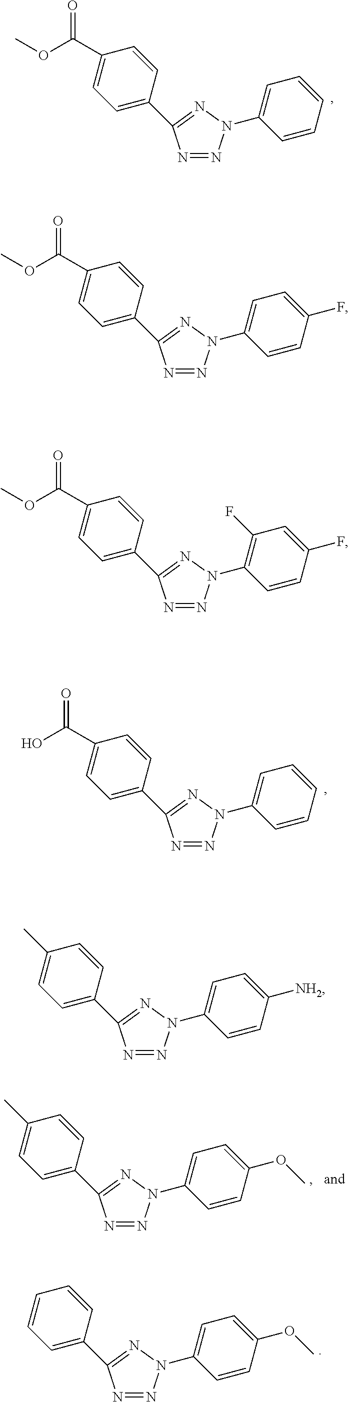

Particularly, the diaryltetrazole compound may be

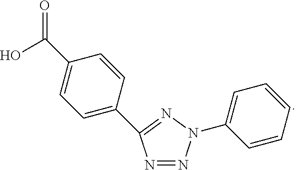 [SMILES: O=C(O)c1ccc(-c2nnn(-c3ccccc3)n2)cc1]

The substituents R₁ to R₁₀ present on the diaryltetrazole compound of formula (I) may affect its detection effectiveness. As demonstrated in examples 1 to 7 below, different diaryltetrazole compounds may show different fluorescent intensity when used to detect the same compound, for instance, acrylic acid. One possible explanation for this is that the substituents R₁ to R₁₀ may be bulky and thus cause steric hindrance when the diaryltetrazole reacts with the acrylic acid or its derivatives thereof. Another possibility is that the substituent groups may have different degrees of ionization while some postulate that not all diaryltetrazole compounds react in the same manner. Accordingly, different degrees of ionization and substitution may lead to varying push-pull effects in the photo-electronics of the pyrazoline fluorophore that may be formed when the diaryltetrazole reacts with the acrylic acid or its derivatives thereof. This varied photo-physical effect leads to different fluorescence intensity. Accordingly, certain effects may dominate and affect the kinetics of the diaryltetrazole reaction with acrylic acid or its derivatives thereof, which may in turn affect the speed of fluorescence emission or intensity.

The probe used in the present method may be biotinylated. Any biotinylating reagents may be attached or conjugated to the probe or to the diaryltetrazole. An example of such a biotinylating reagent may be a biotin. Other biotinylating reagents known to a skilled person may be employed along with the probe used in step (a) of the present method. The advantage of biotinylating the probe or the diaryltetrazole, which is used as the probe, is to enhance the efficiency and accuracy of detection. This is because these biotinylating reagents may bind to specific molecules such as streptavidin, avidin or Neutravidin which may have extremely high affinity, fast on-rate, and high specificity with these biotinylating reagents. Such interactions may help to isolate the biotinylated probe thereby enhancing detection as illustrated in example 14.

Once the probe comprising the diaryltetrazole compound as defined above is mixed with the sample, the sample may be exposed to light as recited in step (b) of the present method. This exposure helps to photoactivate the reaction. This exposure may occur at any wavelength of the electromagnetic spectrum. Any ranges of wavelength falling within the electromagnetic spectrum may also be used. The exposure wavelength may be in the range of 10 nm to 1 mm or any other wavelength falling within this range. Particularly, a wavelength of 302 nm may be used. The use of this wavelength avoids the need for complex light emitting devices.

It may be noted that the present method involves a photoactivated 1,3-dipolar cycloaddition reaction between a diaryltetrazole and an acrylic acid or its derivatives thereof. Upon photo-irradiation at a particular wavelength, for instance 302 nm, the diaryltetrazole may undergo a cycloreversion reaction to generate a highly reactive nitrile imine dipole with the release of nitrogen. This nitrile imine dipole may subsequently react with the acrylic or acrylate dipolarophile to produce a pyrazoline cycloadduct which may emit fluorescence upon photoactivation.

Based on the above, the method may further comprise the step of forming a reactive intermediate. This reactive intermediate may be formed as the diaryltetrazole reaction with acrylic acids or its derivative thereof may be based on the same mechanism as a 1,3-dipolar cycloaddition. This reactive intermediate may be a compound comprising a nitrile imine dipole. Nitrile imines may be classified as a class of organic compounds sharing a common functional group with the general structure R^{x}—CN—NR^{y} corresponding to the conjugate base of an amine bonded to the N-terminus of a nitrile. [Accordingly, R^{x} and R^{y} when used in this context may independently be an optionally substituted organic moiety comprising 1 to 12 carbons or any number of carbon atoms falling within this range. Such an organic moiety may comprise the optional substituents as defined for R₁ to R₁₀.

According to step (c) of the present method, the sample may become fluorescent after exposure to visible light or UV or any other form of photoirradiation. If this is the case, it may indicate the presence of acrylic acid or its derivatives thereof. If no fluorescence is emitted by the sample after photo-activation or exposure to visible light or UV, acrylic acid or its derivatives thereof may be absent from the sample. The fluorescent sample after exposure to light may be attributed to a cycloadduct comprising a fluorescent pyrazoline.

It may be noted that the fluorescent intensity emitted by the pyrazoline cycloadduct may depend on the chemical substituent groups present in the acrylic acid or its derivatives thereof to be detected. The acrylic acid or its derivatives thereof may have electron donating or electron withdrawing chemical substituent groups attached to them, which may affect the diaryltetrazole reaction. These chemical substituent groups may be bulky and cause steric hindrance during the diaryltetrazole reaction.

In the method as defined herein, the acrylic acid or its derivatives thereof may need to be present at a concentration of at least 100 nM, 200 nM, 300 nM, 400 nM or 500 nM before introducing the probe to the sample. The minimal concentration of acrylic acid needed for detection may be at least 100 nM, 200 nM, 300 nM, 400 nM or 500 nM. Meanwhile, the minimal concentration needed for acrylamide to be detected may be 100 nM to 1 μM. The minimal concentration for acrylamide to be detected may be lower or higher than the range of 100 nM to 1 μM. Accordingly, these concentration limits may differ when it comes to detecting other acrylate based derivatives. As for the concentration of diaryltetrazole compound to be used, it may need to be at least 1 nM, 10 μM or any concentration falling between 1 nM to 10 μM. The concentration of diaryltetrazole compound to be used may depend on the concentration of the acrylic acid or its derivatives thereof. Thus, the concentration of the diaryltetrazole compound needed for detection may be less than 10 μM. The concentration of acrylic acid or its derivatives that needs to be available before they can be detected may also depend on the amount of the diaryltetrazole compounds used.

The present method may be conducted over the entire pH range i.e. 1 to 14. The present method may be conducted under acidic, neutral or alkaline conditions. Acidic conditions may occur in the range of pH 1 to 6 while alkaline condition may occur in the range of pH 8 to 14. Neutral conditions may occur at pH 7. Accordingly, any one of steps (a) to (c) may be conducted in any of the pH conditions as described above. Particularly, the exposure of the sample to light in step (b) of the present method may occur under alkaline conditions.

According to the present disclosure, there may be a probe for detecting the presence or absence of acrylic acid or its derivatives thereof in a sample, wherein the probe comprises a diaryltetrazole compound as described above. The diaryltetrazole compound may have the formula

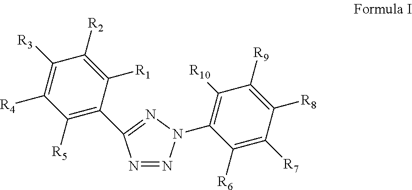

wherein each of R₁ to R₁₀ is independently selected from the group consisting of hydrogen, oxygen, sulfur, hydroxyl, halogen, optionally substituted alkyl, optionally substituted acyl, optionally substituted ester, optionally substituted amino, optionally substituted amine, optionally substituted amide, optionally substituted carboxylic acid, optionally substituted carbonyl, optionally substituted urea, optionally substituted alkoxy, optionally substituted alkyloxy, optionally substituted alkenyl, optionally substituted alkynyl, optionally substituted sulfonamide, optionally substituted aminosulfonamide, optionally substituted sulfonylurea, optionally substituted oxime, optionally substituted cycloalkyl, optionally substituted aryl, optionally substituted heterocycloalkyl, optionally substituted alcohol, optionally substituted heteroalkyl and optionally substituted heteroaryl.

The diaryltetrazole compound may be selected from the group consisting of:

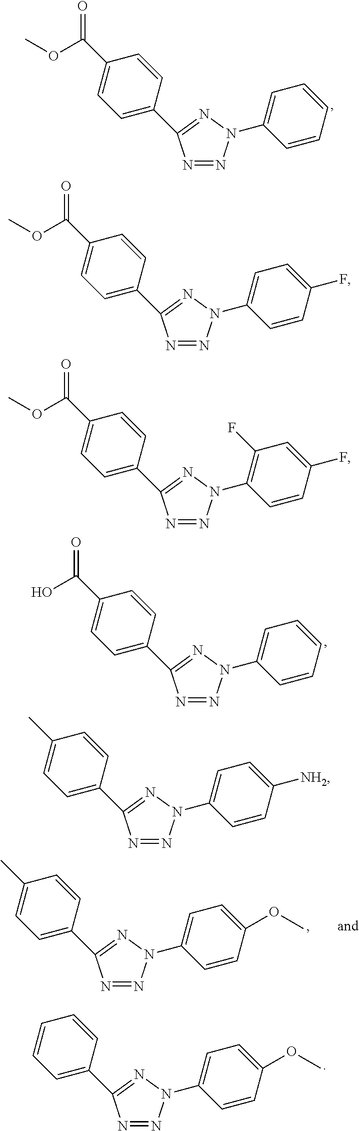

Particularly, as described above, the diaryltetrazole compound may be

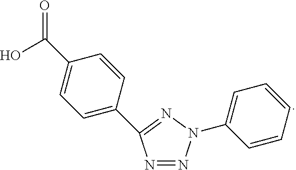 [SMILES: O=C(O)c1ccc(-c2nnn(-c3ccccc3)n2)cc1]

The acrylic acid or its derivatives thereof that may be detected by the present probe are as described above. The probe may be mixed with a sample, wherein the latter may be as defined above. The probe may be used for in vitro or in vivo detection. The sample may or may not comprise acrylic acid or its derivatives thereof. The sample may be a microorganism as defined above. The probe may be biotinylated to enhance detection.

There may be a minimal concentration of the probe needed to detect acrylic acid or its derivatives thereof. This concentration may depend on the amount of the acrylic acid or its derivatives thereof present in the sample. The concentration of the probe in the sample may need to be at least 1 nM, 10 μM or any concentration ranges between 1 nM to 10 μM. The concentration of the probe may depend on the concentration of the acrylic acid or its derivatives thereof that is to be detected. Thus, the concentration of the probe needed for detection may be less than or more than 10 μM. The acrylic acid or its derivative thereof may need to be present at a concentration of at least 100 nM, 200 nM, 300 nM, 400 nM or 500 nM before introducing the probe to the sample. The minimal concentration of acrylic acid needed for detection may be at least 100 nM, 200 nM, 300 nM, 400 nM or 500 nM. Meanwhile, the minimal concentration needed for acrylamide to be detected may be 100 nM to 1 μM per 100 μM of probe or diaryltetrazole used. The minimal concentration needed for acrylamide to be detected may fall between 100 nM to 1 μM or outside this range. The above concentration limits may differ when it comes to detecting other acrylate based derivatives.

The probe comprising the diaryltetrazole compound may emit fluorescence when the diaryltetrazole reacts with the acrylic acid or its derivatives thereof. The intensity of the fluorescence emitted and speed of detection may depend on the factors as discussed above.

The present disclosure also provides the use of a probe as defined above for the detection of the presence or absence of acrylic acid or its derivatives. This probe may comprise the diaryltetrazole compounds as defined above and is thus capable of providing the abovementioned advantages.

According to the present disclosure, there may be a kit comprising the probe as defined above. This kit may enable any user to detect the presence or absence of acrylic acid or its derivatives by contacting the probe with the acrylic acid or its derivatives.

Based on the above disclosure, the present method may be further used to detect a compound containing a terminal alkene comprising the steps of: (1) incubating a sample with a biotinylated probe to form a mixture, (2) irradiating the mixture at an appropriate wavelength to conjugate the biotinylated probe with the terminal-alkene containing compound that may be present in the sample, (3) capturing the conjugates using streptavidin beads, (4) washing the beads thoroughly and eluting the conjugates beads, and (5) measuring the fluorescence of the eluted conjugates to determine the absence or presence of compounds containing a terminal alkene in sample. Magnetic streptavidin beads may be used to aid the isolation or capturing or collection of the conjugated beads.

The compound to be detected as described above may comprise a terminal alkene and such a terminal alkene may include, but not limited to, acrylic acid, acrylamide or acrylate esters etc. A kit for detection of such compounds may be derived by any skilled person on the basis of the above the method as described.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1a depicts the resultant fluorescent emission spectra of diaryltetrazole compound 1 (100 μM with 10 mM of acrylic acid) as exemplified in example 1.

FIG. 1b depicts the resultant fluorescent emission spectra of diaryltetrazole compound 2 (100 μM with 10 mM of acrylic acid) as exemplified in example 2.

FIG. 1c depicts the resultant fluorescent emission spectra of diaryltetrazole compound 3 (100 μM with 10 mM of acrylic acid) as exemplified in example 3.

FIG. 1d depicts the resultant fluorescent emission spectra of diaryltetrazole compound 4 (100 μM with 10 mM of acrylic acid) as exemplified in example 4.

FIG. 1e depicts the resultant fluorescent emission spectra of diaryltetrazole compound 6 (100 μM with 10 mM of acrylic acid) as exemplified in example 6.

FIG. 1f depicts the resultant fluorescent emission spectra of diaryltetrazole compound 7 (100 μM with 10 mM of acrylic acid) as exemplified in example 7.

FIG. 2 depicts the fluorescent emission spectra of the reaction between diaryltetrazole compound 4 and acrylic acid at various concentrations as exemplified in example 9.

FIG. 3 shows the fold increase in fluorescence upon the addition of acrylic acid at various concentrations to 100 μM of diaryltetrazole compound 4 as exemplified in example 9.

FIG. 4 shows the kinetic studies of example 10 concerning the reaction between diaryltetrazole compound 4 (denoted as A) and acrylic acid using HPLC at two UV absorbencies of 254 nm and 370 nm.

FIG. 5 shows the fluorescence emission (turn on) of the reaction mixture containing diaryltetrazole compound 4 and acrylic acid at various time intervals as exemplified in example 10.

FIG. 6a shows the GCMS results of comparative example 1 when the concentration of acrylic acid is at 100 mM.

FIG. 6b shows the magnified GCMS results of comparative example 1 when the concentration of acrylic acid is at 100 mM.

FIG. 6c shows the mass spectrometry data of the acrylic acid peak when the concentration of acrylic acid is at 100 mM.

FIG. 7a shows the GCMS results of comparative example 1 when the concentration of acrylic acid is at 10 mM.

FIG. 7b shows the magnified GCMS results of comparative example 1 when the concentration of acrylic acid is at 10 mM.

FIG. 8a shows the GCMS results of comparative example 1 when the concentration of acrylic acid is at 1 mM.

FIG. 8b shows the magnified GCMS results of comparative example 1 when the concentration of acrylic acid is at 1 mM.

FIG. 9a shows the GCMS results of comparative example 1 when the concentration of acrylic acid is at 750 μM.

FIG. 9b shows the magnified GCMS results of comparative example 1 when the concentration of acrylic acid is at 750 μM.

FIG. 10a shows the GCMS results of comparative example 1 when the concentration of acrylic acid is at 500 μM.

FIG. 10b shows the magnified GCMS results of comparative example 1 when the concentration of acrylic acid is at 500 μM.

FIG. 11a shows the GCMS results of comparative example 1 when the concentration of acrylic acid is at 250 μM.

FIG. 11b shows the magnified GCMS results of comparative example 1 when the concentration of acrylic acid is at 250 μM.

FIG. 12a shows the GCMS results of comparative example 1 when the concentration of acrylic acid is at 100 μM.

FIG. 12b shows the magnified GCMS results of comparative example 1 when the concentration of acrylic acid is at 100 μM.

FIG. 13a shows the GCMS results of comparative example 1 when the concentration of acrylic acid is at 10 μM.

FIG. 13b shows the magnified GCMS results of comparative example 1 when the concentration of acrylic acid is at 10 μM.

FIG. 14a shows the GCMS results of comparative example 1 when no acrylic acid is present.

FIG. 14b shows the magnified GCMS results of comparative example 1 when no acrylic acid is present.

FIG. 15a shows the fluorescence assay results of example 11 concerning acrylic acid standards in Lysogeny broth (LB) media.

FIG. 15b shows the fluorescence assay results of example 11 concerning acrylic acid standards in minimum media.

FIG. 16a shows the pH dependence of the diaryltetrazole reaction with acrylic acid based on example 12.

FIG. 16b shows the pH dependence of the diaryltetrazole reaction with acrylamide based on example 12.

FIG. 17 compares the fluorescence measurements of acrylic acid and two different grades of acrylamide at different pH as shown in example 13.

FIG. 18 compares the fluorescence results of acrylamide detected at various concentrations as shown in example 13.

FIG. 19 compares the fluorescence results of acrylamide detected in complex organic/detergent mixture under the presence of different oil media as shown in example 13.

FIG. 20 compares the fluorescence results of acrylamide detected in different oil media as shown in example 13.

FIG. 21 shows the pH dependence of the fluorescent probe for acrylic acid and acrylamide as shown in example 13.

FIG. 22a shows the fluorescence measurements of example 14 concerning various concentrations of acrylamide using biotinylated probe.

FIG. 22b shows the fluorescence measurements of example 14 concerning various concentrations of acrylamide using unbiotinylated probe.

FIG. 22c shows the isolation effects of using streptavidin beads on biotinylated and unbiotinylated probes as shown in example 14.

FIG. 23 shows the fluorescence measurements of example 14 concerning various concentrations of acrylamide using biotinylated probe.

FIG. 24 shows the detection of acrylic acid in E. coli using compound 4 as exemplified in example 15. A small bar has been indicated in the bottom rightmost picture which represents a scale bar of 10 μm (see DIC image at the bottom right of FIG. 24).

FIG. 25a depicts the detection of acrylic acid (and/or the reaction intermediates) present in Clostridium propionicum grown in media containing 5 mM 3-butynoic acid either untreated, treated with acrylic acid, diaryltetrazole compound 4 or both as exemplified in example 15. A small bar has been indicated in the bottom rightmost picture which represents a scale bar of 2 μm (see DAPI image at the bottom right of FIG. 25a).

FIG. 25b shows the fluorescence signals of acrylic acid detected from the cell lysates of the experiment as shown in FIG. 25a that were quantitatively measured using a fluorescence plate reader.

FIG. 26 shows the fluorescence signal of acrylic acid detected from bacterial cell lysates from Clostridium propionicum and E. Cali grown in medium either not treated (26a) or treated with 5 and 10 mM 3-butynoic acid (26b and 26c respectively).

### EXAMPLES

Non-limiting examples of the invention and a comparative example will be further described in greater detail by reference to specific Examples, which should not be construed as in any way limiting the scope of the invention.

### Synthetic Details of Diaryltetrazoles and Characterization of Pyrazoline Product

All chemicals and solvents were purchased from commercial sources and used directly without purification. Flash chromatography was performed using SiliCycle P60 silica gel (40-63 μm, 60 Å). ¹H NMR spectra were recorded with Bruker Avance III 400, and chemical shifts were reported in ppm using either TMS or deuterated solvents as internal standards (TMS, 0.00; CDCl₃, 7.26; C₆D₆, 7.15; DMSO-d₆, 2.50). Multiplicity was reported as the follows: s=singlet, d=doublet, t=triplet, q=quartet, m=multiplet, b=broad. ¹³C NMR spectra were recorded at 75.4 MHz, and chemical shifts were reported in ppm using the deuterated solvents as internal standards (CDCl₃, 77.0; DMSO-d₆, 39.5; C₆D₆, 128.0). LC-MS analysis was performed using Waters 3100 Single Quadrupole LCMS System. Kinetic studies were performed using Phenomenex Kinetex 2.6u XB-C18 column (50×4.6 mm) Flow rate was 1 mL/min and UV detection was set at 254 and 370 nm as indicated. Gas chromatography was carried out on a Shimadzu GCMS QP 2010 using a 30.0 m×0.25 mm, inner diameter of 0.25 μm HP-INNOWAX column which was programmed from 40 to 250° C. at 10° C./min. Fluorescence detection were performed with Tecan Infinite M1000.

### Example 1—Synthesis and Characterization of Diaryltetrazole Compound 1 and Pyrazoline Product 1P

Reaction Scheme 1a below shows the reaction pathway of diaryltetrazole compound 1.

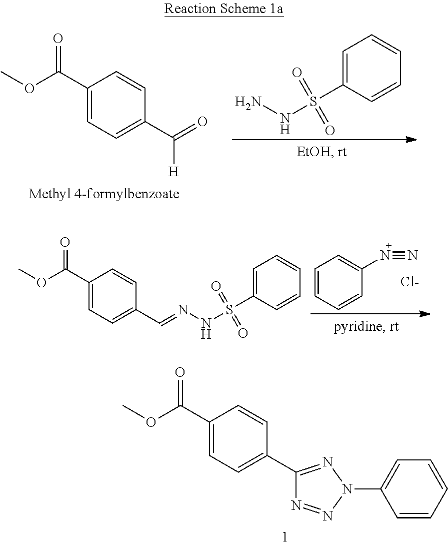

Methyl 4-formylbenzoate (0.824 g, 5 mmol) was dissolved in ethanol (50 mL), and benzenesulfonohydrazine (0.863 g, 5 mmol) was added. The mixture was stirred at room temperature for 1 hour, then quenched with water (100 mL) and stirred for 15 minutes at room temperature. The precipitate was filtered and washed with cold ethanol. The precipitate was then dissolved in pyridine (30 mL) for the next reaction. Aniline (0.465 g, 0.46 mL, 5 mmol) was separately dissolved in water:ethanol (1:1, 8 mL) and concentrated HCl (1.3 mL) was added. NaNO₂ (0.346 g, 5 mmol) was also separately dissolved in water (2 mL). The aniline solution was cooled in an ice bath for 5 minutes before addition of NaNO₂ solution to the aniline solution drop wise in an ice bath. The reaction mixture was added dropwise to the cooled product from the first reaction in an ice bath. The reaction mixture was stirred for 1 hour at room temperature. Extraction was then carried out with ethyl acetate (100 mL×3). 3 M HCl (250 mL) was added to the combined organic layers and stirred vigorously for 10 minutes. The organic layer was concentrated and the product was precipitated with hexane. The product was further washed with cold hexane. The hexane was incubated in ice before being used for washing the product. The temperature of the incubated hexane used for washing is about 0° C. to 10° C. This method of cooling the temperature of the hexane has been repeated in the subsequent examples. to obtain a pale orange solid (0.538 g, 38%). ¹H NMR (400 MHz, Chloroform-d) δ 8.35 (dd, J=8.2, 0.6 Hz, 2H), 8.24-8.18 (m, 4H), 7.63-7.56 (m, 2H), 7.56-7.50 (m, 1H), 3.97 (s, 3H). ¹³C NMR (101 MHz, CDCl₃) δ 166.52, 164.41, 136.87, 131.92, 131.28, 130.24, 129.89, 129.75, 127.01, 119.97, 52.32. HRMS (ESI) calculated for C₁₅H₁₂N₄O₂ 280.096 [M+H+], found 280.096.

Reaction Scheme 1b below shows the reaction pathway of pyrazoline product 1P.

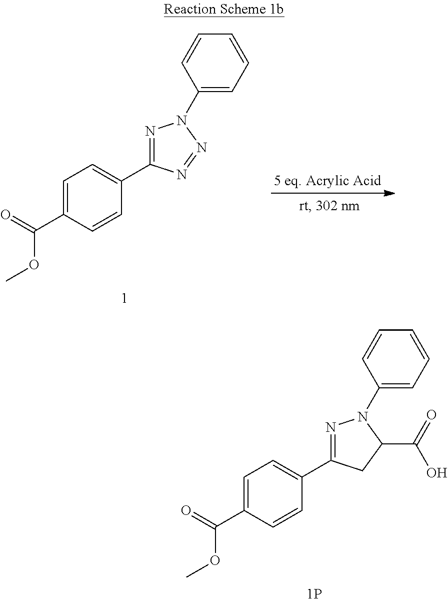 [SMILES: COC(=O)c1ccc(-c2nnn(-c3ccccc3)n2)cc1 ; COC(=O)c1ccc(C2=NN(c3ccccc3)C(C(=O)O)C2)cc1]

A 4 mL ethyl acetate (EA) solution of diaryltetrazole compound 1 (20 mg, 0.0713 mmol) and acrylic acid (24.5 μL, 5 equivalent molar) were irradiated with 302 nm UV lamp for 3 hour. The excess solvent and reagent were removed by reduced pressure to produce crude product which was subsequently purified by silica gel column chromatography. (Hexane (Hex):EA, 1:1) Product 1P was collected as yellow solid. ¹H NMR (400 MHz, HRMS (ESI) calculated for C₁₈H₁₆N₂O₄ 324.1115 [M+H+], found 324.111.

### Example 2—Synthesis and Characterization of Diaryltetrazole Compound 2 and Pyrazoline Product 2P

Reaction Scheme 2a below shows the reaction pathway of diaryltetrazole compound 2.

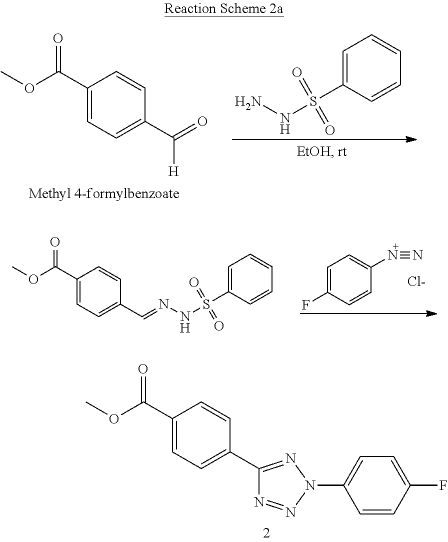

Methyl 4-formylbenzoate (0.820 g, 5 mmol) was dissolved in ethanol (50 mL), followed by addition of benzenesulfonohydrazine (0.862 g, 5 mmol). The mixture was stirred at room temperature for 1 hour, then quenched with water (100 mL) and stirred for 15 minutes at room temperature. The precipitate was filtered, washed with cold ethanol and dissolved in pyridine (30 mL) to form solution A. 4-fluoroaniline was then dissolved (0.555 g, 0.48 mL, 5 mmol) in water:ethanol (1:1, 8 mL) and concentrated HCl (1.3 mL). NaNO₂ was dissolved (0.345 g, 5 mmol) in water (2 mL). Both mixtures were cooled in ice bath for 5 minutes before addition of NaNO₂ solution to 4-fluoroaniline solution drop wise in ice bath to form solution B. Solution B was added to solution A drop wise in ice bath. The mixture was then stirred for 1 hour at room temperature. The mixture was extracted with ethyl acetate (100 mL×3). 3M HCl (250 mL) was added to combine organic layer, followed by stirring vigorously for 10 minutes. The organic layer was concentrated and product was precipitated with hexane. The product was washed with cold hexane to obtain pale pink solid (0.777 g, 52%)¹H NMR (400 MHz, Chloroform-d) δ 8.36 8.29 (m, 2H), 8.24-8.16 (m, 4H), 7.29 (dd, J=9.1, 7.9 Hz, 2H), 3.97 (s, 3H). ¹³C NMR (101 MHz, CDCl₃) δ 166.46, 164.49, 164.40, 161.91, 131.97, 131.07, 130.24, 126.97, 121.96, 121.87, 116.90, 116.67, 52.33. HRMS (ESI) calculated for C₁₅H₁₁FN₄O₂ 298.0865 [M+H+], found 298.0866.

Reaction Scheme 2b below shows the reaction pathway of pyrazoline product 2P.

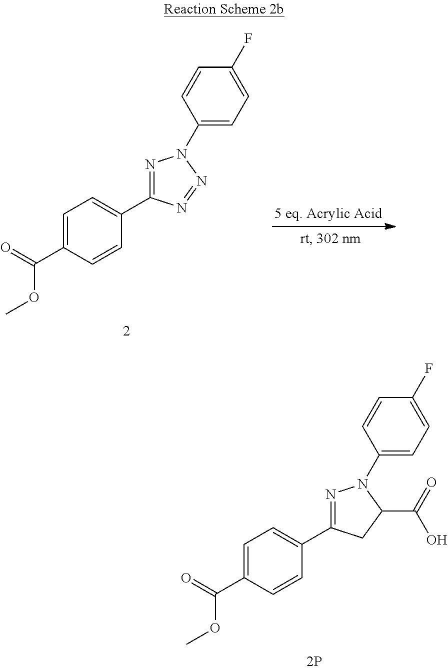 [SMILES: COC(=O)c1ccc(-c2nnn(-c3ccc(F)cc3)n2)cc1 ; COC(=O)c1ccc(C2=NN(c3ccc(F)cc3)C(C(=O)O)C2)cc1]

A 4 mL ethyl acetate solution of diaryltetrazole compound 2 (20 mg, 0.0671 mmol) and acrylic acid (23 μL, 5 equivalent molar) were irradiated with 302 nm UV lamp for 3 hour. The excess solvent and reagent were removed by reduced pressure to produce crude product which was subsequently purified by silica gel column chromatography. (Hex:EA, 1:1) Product 2P was collected as yellow solid. ¹H NMR (400 MHz, HRMS (ESI) calculated for C₁₈H₁₅FN₂O₄ 342.1022 [M+H+], found 342.1016.

### Example 3—Synthesis and Characterization of Diaryltetrazole Compound 3 and Pyrazoline Product 3P

Reaction Scheme 3a below shows the reaction pathway of diaryltetrazole compound 3.

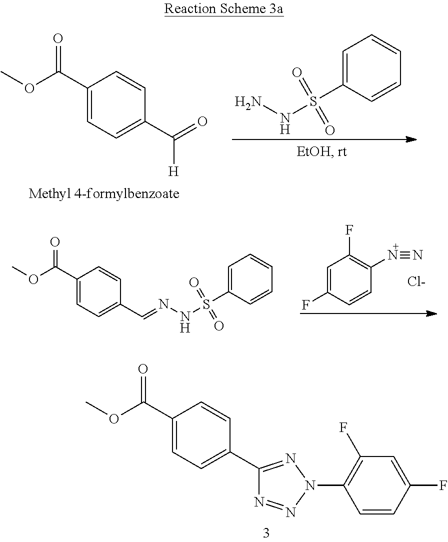

Methyl 4-formylbenzoate (0.820 g, 5 mmol) was dissolved in ethanol (50 mL), followed by addition of benzenesulfonohydrazine (0.859 g, 5 mmol). The mixture was stirred at room temperature for 1 hour, then quenched with water (100 mL) and stirred for 15 minutes at room temperature. The precipitate was filtered, washed with cold ethanol and dissolved in pyridine (30 mL) to form solution A. 2, 4-fluoroaniline was then dissolved (0.645 g, 0.50 mL, 5 mmol) in water:ethanol (1:1, 8 mL) and concentrated HCl (1.3 mL). NaNO₂ was dissolved (0.345 g, 5 mmol) in water (2 mL). Both mixtures were cooled in ice bath for 5 minutes before addition of NaNO₂ solution to 2, 4-fluoroaniline solution drop wise in ice bath to form solution B. Solution B was added to solution A drop wise in ice bath. The mixture was then stirred for 1 hour at room temperature. The mixture was extracted with ethyl acetate (100 mL×3). 3M HCl (250 mL) was added to combine organic layer and stirred vigorously for 10 minutes. The organic layer was concentrated and the product was precipitated with hexane. The product was washed with cold hexane to obtain red solid (0.173 g, 11%). ¹H NMR (400 MHz, Chloroform-d) δ 8.35 8.30 (m, 2H), 8.23-8.18 (m, 2H), 7.91 (td, 0.1=8.6, 5.6 Hz, 1H), 7.19-7.10 (m, 2H), 3.97 (s, 3H). ¹³C NMR (101 MHz, CDCl₃) δ 206.88, 129.90, 129.11, 127.89, 127.14, 77.32, 77.20, 77.00, 76.68, 30.89. HRMS (ESI) calculated for C₁₅H₁₀F₂N₄O₂ 316.0765 [M+H+], found 316.0772.

Reaction Scheme 3b below shows the reaction pathway of pyrazoline product 3P.

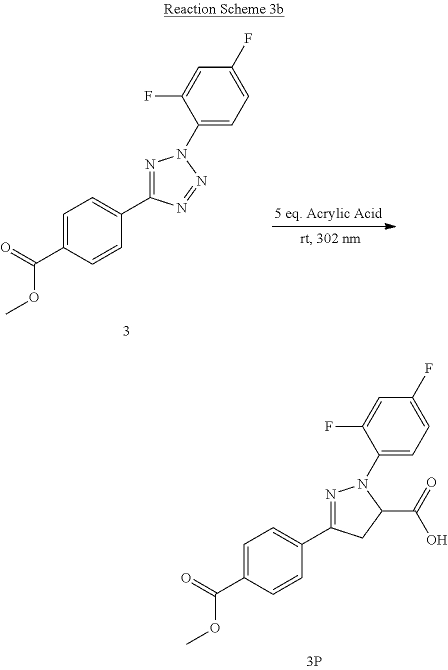 [SMILES: COC(=O)c1ccc(-c2nnn(-c3ccc(F)cc3F)n2)cc1 ; COC(=O)c1ccc(C2=NN(c3ccc(F)cc3F)C(C(=O)O)C2)cc1]

A 4 mL ethyl acetate solution of diaryltetrazole compound 3 (20 mg, 0.0633 mmol) and acrylic acid (21.7 μL, 5 equivalent molar) were irradiated with 302 nm UV lamp for 3 hour. The excess solvent and reagent were removed by reduced pressure to produce the crude product which was subsequently purified by silica gel column chromatography. (Hexane:EA, 1:1) Product 3P was collected as yellow solid. ¹H NMR (400 MHz, HRMS (ESI) calculated for C₁₈H₁₄F₂N₂O₄ 360.0929 [M+H+], found 360.0922.

### Example 4—Synthesis and Characterization of Diaryltetrazole Compound 4 and Pyrazoline Product 4P

Reaction Scheme 4a below shows the reaction pathway of diaryltetrazole compound 4.

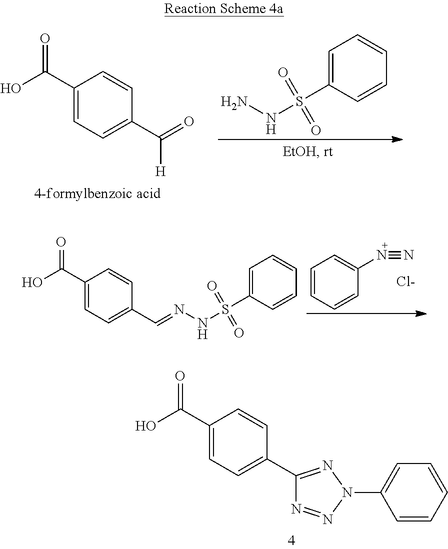

4-formylbenzoic acid (1.000 g, 6 mmol) was dissolved in ethanol (100 mL), followed by addition of benzenesulfonohydrazine (1.160 g, 6 mmol). The mixture was stirred at room temperature for 1 hour, then quenched with water (100 mL) and stirred for 15 minutes at room temperature. Precipitate was filtered, washed with cold ethanol and dissolved in pyridine (30 mL) to form solution A. Aniline was then dissolved (0.587 g, 0.60 mL, 5 mmol) in water:ethanol (1:1, 8 mL) and concentrated HCl (1.3 mL). NaNO₂ was dissolved (0.455 g) in water (2 mL). Both mixtures were cooled in ice bath for 5 minutes before addition of NaNO₂ solution to aniline solution drop wise in ice bath to form solution B. Solution B was added to solution A drop wise in ice bath. Mixture was then stirred for 1 hour at room temperature. The mixture was extracted with ethyl acetate (100 mL×3). 3M HCl (250 mL) was added to combine organic layer and stirred vigorously for 10 minutes. The solvent was removed and then dissolved in dichloromethane. The product was precipitated with hexane. The product was washed with cold hexane to obtain red solid (0.820 g, 45%). ¹H NMR (400 MHz, Methanol-d4) δ 8.39 8.34 (in, 21-1), 8.27-8.22 (m, 4H), 7.71-7.66 (m, 2H), 7.63 (d, J=7.3 Hz, 1H). ¹³C NMR (101 MHz, MeOD) δ 147.01, 141.50, 141.48, 129.93, 129.60, 127.95, 127.89, 127.44, 127.41, 125.52, 119.62. HRMS (ESI) calculated for C₁₄H₁₀N₄O₂ 266.0805 [M+H+], found 266.0804.

Reaction Scheme 4b below shows the reaction pathway of pyrazoline product 4P.

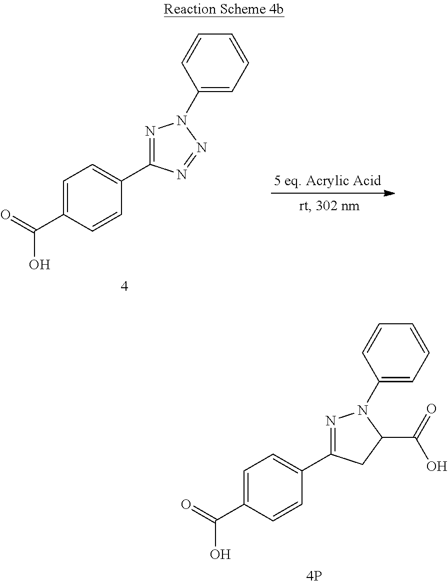 [SMILES: O=C(O)c1ccc(-c2nnn(-c3ccccc3)n2)cc1 ; O=C(O)c1ccc(C2=NN(c3ccccc3)C(C(=O)O)C2)cc1]

A 4 mL dichloromethane and methanol solution of diaryltetrazole compound 4 (20 mg, 0.0751 mmol) and acrylic acid (25.8 μL, 5 equivalent molar) were irradiated with 302 nm UV lamp for 3 hour. The excess solvent and reagent were removed by reduced pressure to produce crude product which was subsequently purified by silica gel column chromatography. (MeOH:DCM, 3:17) Product 4P was collected as yellow solid. ¹H NMR (400 MHz, HRMS (ESI) calculated for C₁₇H₁₄N₂O₄ 310.0948 [M+H+], found 310. 0954.

### Example 5—Synthesis and Characterization of Diaryltetrazole Compound 5

Reaction Scheme 5a below shows the reaction pathway of diaryltetrazole compound 5.

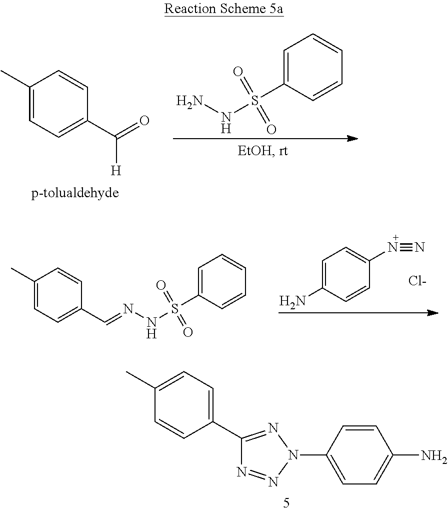

p-tolualdehyde (1.000 g, 8 mmol) was dissolved in ethanol (60 mL), followed by addition of benzenesulfonohydrazine (1.433 g, 8 mmol). The mixture was stirred at room temperature for 1 hour, then quenched with water (100 mL) and stirred for 15 minutes at room temperature. The precipitate was filtered, washed with cold ethanol and dissolved in pyridine (30 mL) to form solution A. 1,4-phenylenediamine was then dissolved (0.905 g, 8 mmol) in water:ethanol (1:1, 10 mL) and concentrated HCl (1.3 mL). NaNO₂ was dissolved (0.583 g, 8 mmol) in water (2 mL). Both mixtures were cooled in ice bath for 5 minutes before addition of NaNO₂ solution to 1,4-phenylenediamine solution drop wise in ice bath to form solution B. Solution B was added to solution A drop wise in ice bath. The mixture was then stirred for 1 hour at room temperature. Mixture was extracted with Ethyl Acetate (100 mL×3). 3M HCl (250 mL) was added to combine organic layer and stirred vigorously for 10 minutes. The organic layer was concentrated to obtain a red solid. The crude product was purified with column chromatography (Hex:EA 1:1) to collect as yellow solid (1.149 g, 54.9%). ¹H NMR (400 MHz, Chloroform-d) δ 8.47-8.39 (m, 1H), 8.02-7.97 (m, 2H), 7.76 (s, 1H), 7.54 (d, J=7.2 Hz, 1H), 7.51-7.46 (m, 2H), 7.46-7.42 (m, 2H), 7.12 (d, J=7.9 Hz, 2H), 2.32 (s, 3H). ¹³C NMR (101 MHz, CDCl₃) δ 148.27, 140.92, 138.32, 133.17, 130.26, 129.34, 128.93, 127.85, 127.31, 29.62, 21.39. HRMS (ESI) calculated for C₁₄H₁₃N₅ 251.1161 [M+H+], found 251.1171.

Reaction Scheme 5b below shows the reaction pathway of pyrazoline product 5P.

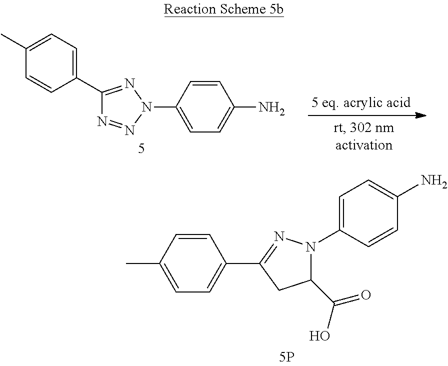 [SMILES: Cc1ccc(-c2nnn(-c3ccc(N)cc3)n2)cc1 ; Cc1ccc(C2=NN(c3ccc(N)cc3)C(C(=O)O)C2)cc1]

No fluorescence appears to be observed when compound 5 in used. This may be due to internal quenching of fluorescence possibly caused by the presence of the amine functional group on one of the aryl groups of the diaryltetrazole Amine groups are known to be capable of participating in intramolecular photoinduced electron transfer (PET), which is a possible mechanism for causing quenching of fluorescence.

### Example 6—Synthesis and Characterization of Diaryltetrazole Compound 6 and Pyrazoline Product 6P

Reaction Scheme 6a below shows the reaction pathway of diaryltetrazole compound 6.

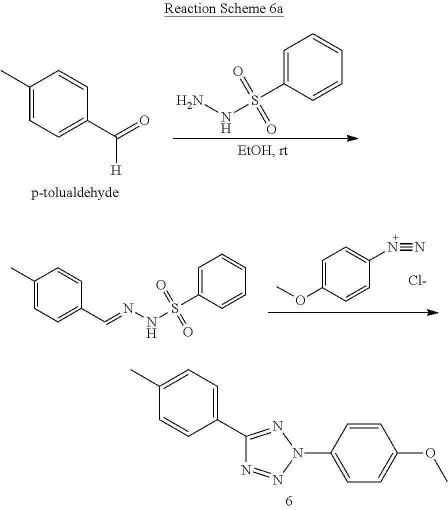

p-tolualdehyde (1.000 g, 8 mmol) was dissolved in ethanol (60 mL), followed by addition of benzenesulfonohydrazine (1.433 g, 8 mmol). The mixture was stirred at room temperature for 1 hour, then quenched with water (100 mL) and stirred for 15 minutes at room temperature. Precipitate was filtered, washed with cold ethanol and dissolved in pyridine (30 mL) to form solution A. 4-methoxyaniline was then dissolved (1.067 g, 8 mmol) in water:ethanol (1:1, 10 mL) and concentrated HCl (1.3 mL). NaNO₂ was dissolved (0.583 g, 8 mmol) in water (2 mL). Both mixtures were cooled in ice bath for 5 minutes before addition of NaNO₂ solution to 4-methoxyaniline solution drop wise in ice bath to form solution B. Solution B was added to solution A drop wise in ice bath. The mixture was then stirred for 1 hour at room temperature. The mixture was extracted with ethyl acetate (100 mL×3). 3M HCl (250 mL) was added to combine organic layer and stirred vigorously for 10 minutes. The organic layer was concentrated to obtain a red solid. The crude product was purified with column chromatography (Hex:EA 5:1) to obtain a orange red solid (0.8921 g, 41.6%). ¹H NMR (400 MHz, Chloroform-d) δ 8.11 (d, J=8.2 Hz, 2H), 8.09-8.06 (m, 2H), 7.32-7.29 (m, 2H), 7.05-7.01 (m, 2H), 3.86 (s, 3H), 2.41 (s, 3H). ¹³C NMR (101 MHz, CDCl₃) δ 165.05, 160.46, 140.62, 129.62, 126.90, 121.37, 114.69, 55.73, 21.53. HRMS (ESI) calculated for C₁₅H₁₄N₄O 266.1166 [M+H+], found 266.1168.

Reaction Scheme 6b below shows the reaction pathway of pyrazoline product 6P.

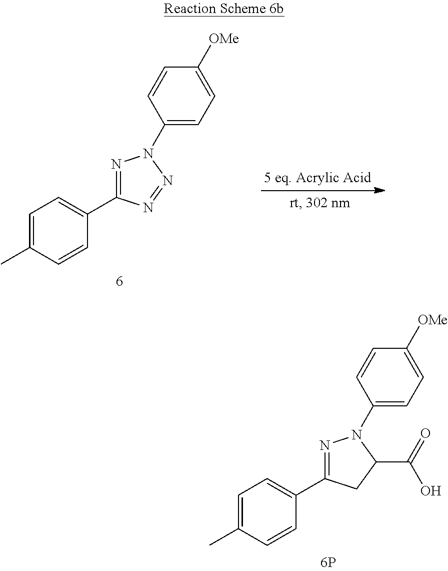 [SMILES: COc1ccc(-n2nnc(-c3ccc(C)cc3)n2)cc1 ; COc1ccc(N2N=C(c3ccc(C)cc3)CC2C(=O)O)cc1]

A 4 mL ethyl acetate solution of diaryltetrazole compound 6 (20 mg, 0.0751 mmol) and acrylic acid (25.7 μL, 5 equivalent molar) were irradiated with 302 nm UV lamp for 3 hour. The excess solvent and reagent were removed by reduced pressure to produce crude product which was subsequently purified by silica gel column chromatography. (MeOH:DCM, 1:20) Product 6P was collected as yellow solid. HRMS (ESI) calculated for C₁₈H₁₈N₂O₃ 310.1325 [M+H+], found 310.1317.

### Example 7—Synthesis and Characterization of Diaryltetrazole Compound 7 and Pyrazoline Product 7P

Reaction Scheme 7a below shows the reaction pathway of diaryltetrazole compound 7.

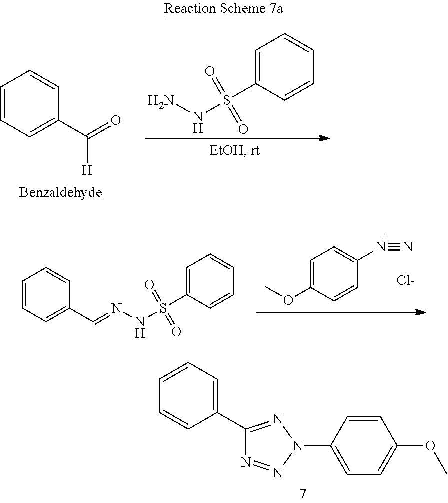

Benzaldehyde (1.000 g, 8 mmol) was dissolved in ethanol (60 mL), followed by addition of benzenesulfonohydrazine (1.623 g, 8 mmol). The mixture was stirred at room temperature for 1 hour, then quenched with water (100 mL) and stirred for 15 minutes at room temperature. Precipitate was filtered, washed with cold ethanol and dissolved in pyridine (30 mL) to form solution A. 4-methoxyaniline was then dissolved (1.067 g, 8 mmol) in water:ethanol (1:1, 10 mL) and concentrated HCl (1.3 mL). NaNO₂ was dissolved (0.583 g, 8 mmol) in water (2 mL). Both mixtures were cooled in ice bath for 5 minutes before addition of NaNO₂ solution to 4-methoxyaniline solution drop wise in ice bath to form solution B. Solution B was added to solution A drop wise in ice bath. Mixture was then stirred for 1 h at room temperature. The mixture was extracted with ethyl acetate (100 mL×3). 3M HCl (250 mL) was added to combine organic layer and stirred vigorously for 10 minutes. The organic layer was concentrated to obtain a red solid. The crude product was ran through column chromatography (DCM: MeOH 9:1) to obtain a red solid (1.420 g, 59.7%). ¹H NMR (400 MHz, Chloroform-d) δ 8.23 (dd, J=7.9, 1.7 Hz, 2H), 8.09 (d, J=9.1 Hz, 2H), 7.53-7.47 (m, 3H), 7.04 (d, J=9.1 Hz, 2H), 3.87 (s, 3H). ¹³C NMR (101 MHz, CDCl₃) δ 163.68, 159.20, 129.10, 127.60, 125.68, 120.09, 113.37, 54.35. HRMS (ESI) calculated for C₁₄H₁₂N₄O 252.1003 [M+H+], found 252.1011.

Reaction Scheme 7b below shows the reaction pathway of pyrazoline product 7P.

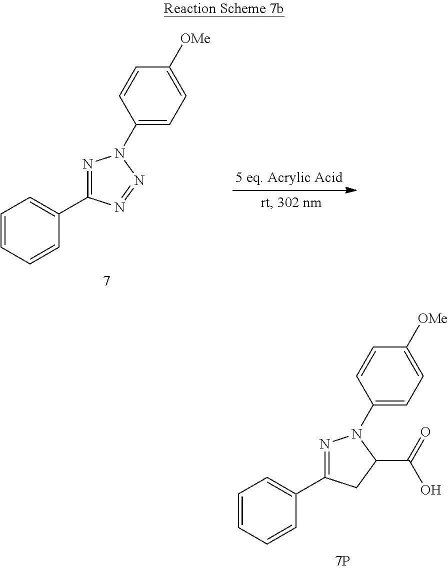 [SMILES: COc1ccc(-n2nnc(-c3ccccc3)n2)cc1 ; COc1ccc(N2N=C(c3ccccc3)CC2C(=O)O)cc1]

A 4 mL ethyl acetate solution of diaryltetrazole compound 7 (20 mg, 0.0793 mmol) and acrylic acid (27.2 μL, 5 equivalent molar) were irradiated with 302 nm UV lamp for 3 hour. The excess solvent and reagent were removed by reduced pressure to produce crude product which was subsequently purified by silica gel column chromatography. (MeOH:DCM, 1:20) Product 7P was collected as yellow solid. ¹H NMR (400 MHz, HRMS (ESI) calculated for C₁₇H₁₆N₂O₃ 296.1169 [M+H+], found 296.1161.

### Results of Examples 1 to 7

Examples 1 to 7 demonstrate the sensitivity and throughput of the presently described fluorescence assay method for detection of acrylic acid. The present method may utilize the photo-inducible bio-orthogonal chemistry, which involves a photoactivated 1,3-dipolar cycloaddition reaction between a diaryltetrazole and an acrylic acid or its derivatives thereof. This may or may not further extend to alkene.

Upon photo-irradiation at 302 nm, the diaryltetrazole undergoes a cyclo-reversion reaction, generating a highly reactive nitrile imine dipole and releases N₂. This nitrile imine dipole may react with the dipolarophile to produce a pyrazoline cycloadduct, which is capable of being fluorescent. The seven diaryltetrazoles as synthesized above have been tested for their ability to detect the presence or absence of acrylic acid. Compounds 5, 6 and 7 are designed to incorporate electron-donating groups on the aryl rings which tend to increase the rate of reaction. The presence of electron donating substituents in the N-phenyl ring may lead to an increase in the reaction rate due to the highest occupied molecular orbital-lifting effect (HOMO-lifting effect). The rate of the cycloaddition reaction is capable of being accelerated when the HOMO energy level of the nitrile imine dipole is increased. Fluorescence results for the reaction between the seven diaryltetrazole compounds with acrylic acid are indicated in table 1 below.

TABLE 1

| | | |
|---|---|---|
| | | Fold increase |
| λ_{ex,max} | λ_{em,max} | in fluorescence |

| Compound^{a} | (nm) | (nm) | 10 mM | 100 μM |
|---|---|---|---|---|
| 1 | 395 | 482 | 3.23 | N.A. |
| 2 | 392 | 487 | 3.38 | 1.12 |
| 3 | 380 | 482 | 3.17 | 1.12 |
| 4 | 380 | 520 | 5.15 | 132 |
| 5 | N.A. | N.A. | N.A. | N.A. |
| 6 | 371 | 450, 475 | 3.87 | N.A. |
| 7 | 368 | 456, 475 | 3.38 | 1.43 |

^{a}represents that 100 μM of each compound was reacted with either 10 mM or 100 μM of acrylic acid. Photoirradiation of 1 minute was carried out at 302 nm.

All seven compounds were reacted with acrylic acid and their fluorescence properties were measured. The results are summarized in Table 1 above. Compounds 1, 2, 3, 6 and 7 gave moderate fluorescence increases of around 3-fold above the background. Compound 5 appears to be unreactive towards acrylic acid, forming little or no fluorescent product. Interestingly, compound 4 gave the highest fluorescence turn on signal (132-fold increase) upon photoactivation in the presence of 100 μM of acrylic acid. The lower limit of detection of acrylic acid for compound 4 was 500 nM upon 1 minute photoactivation period with UV light at 302 nm. Accordingly, the diaryltetrazole compounds of the present disclosure are capable of being used to detect the presence or absence of acrylic acids or its derivatives thereof. As mentioned above, no fluorescence appears to be exhibited by compound 5 as there may be internal quenching of fluorescence due to the presence of the amine functional group on one of the aryl groups of the diaryltetrazole Amine groups are known to be capable of participating in intramolecular photoinduced electron transfer (PET), which is a mechanism that is capable of causing quenching of fluorescence.

### Example 8—Fluorescence Characterization of the Reaction Between Diaryltetrazoles and Acrylic Acid of Examples 1 to 7

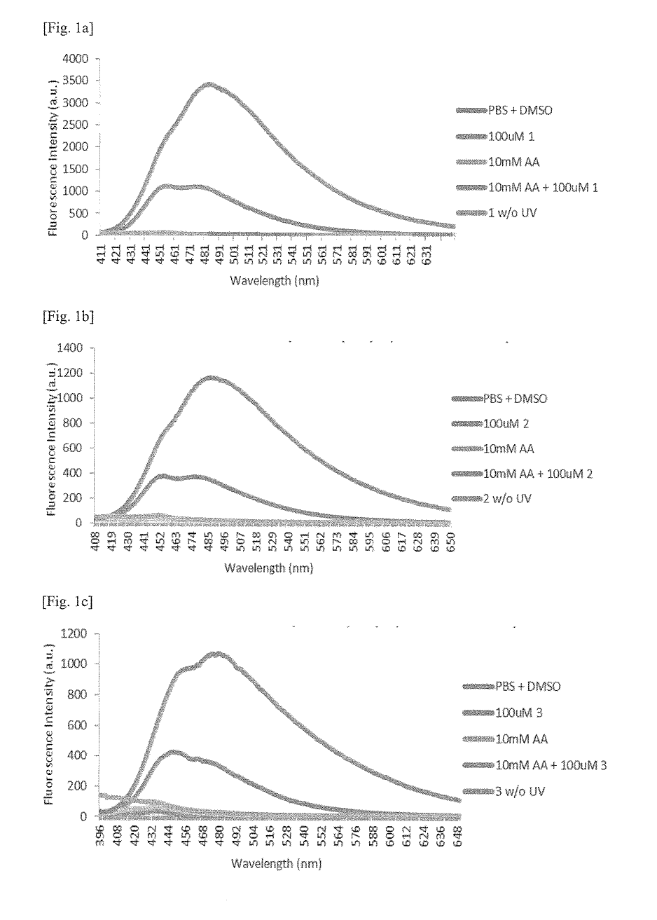

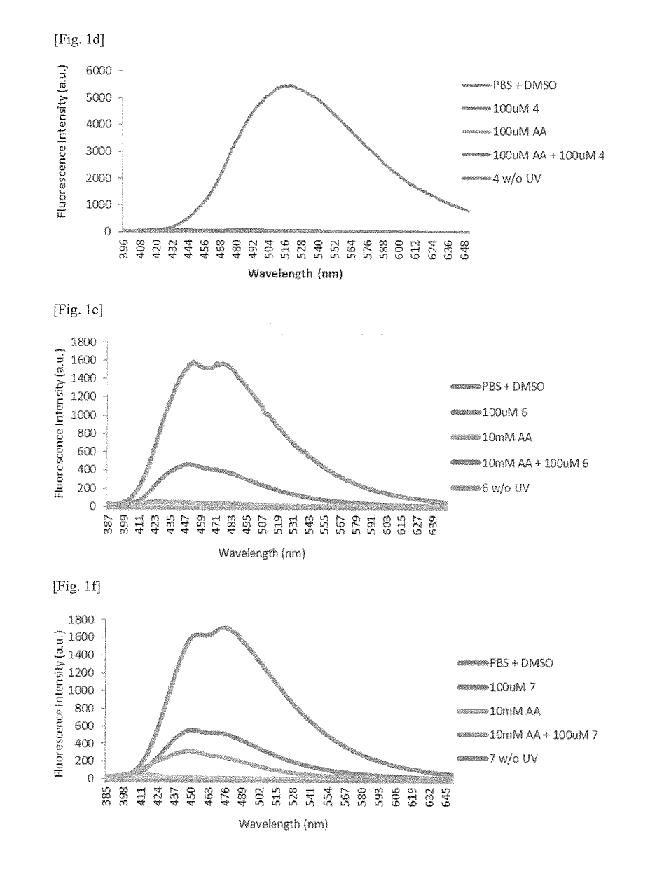

A solution containing the diaryltetrazole (0.022 mmol) and acrylic acid (0.02 to 0.050 mmol) was irradiated with a hand-held 302-nm UV lamp for 1 minute. Experiments were performed in a black 96-well plate with a total reaction volume of 100 The maximum excitation wavelengths indicated in Table 1 were used for each of the scans. The fluorescence emission spectrums of the reaction are shown in FIG. 1a to if using 10 μM of acrylic acid. The spectra for control experiments where either of the reactants was omitted have also been depicted in each of FIGS. 1a to 1f. Accordingly, no fluorescence turn on signal was observed for control experiments. Observably, no fluorescence turn on was observed in the absence of UV activation. As can be seen in FIG. 1a to 1f, only the spectrum for the wells containing both the diaryltetrazole compounds and the acrylic acid showed the highest intensity curve when photoactivated.

### Example 9—Limit of Detection of Diaryltetrazole Compound 4

As mentioned earlier, the lower limit of detection of acrylic acid for compound 4 was 500 nM of acrylic acid upon a 1 minute photoactivation period with UV light at 302 nm. Table 2 below shows the concentration and its corresponding increase in fluorescence.

TABLE 2

| Concentration of compound 4 | 100 μM | 100 μM | 100 μM | 100 μM |
|---|---|---|---|---|
| Conc. of acrylic acid | 100 μM | 10 μM | 1 μM | 500 nM |
| Fold increase in fluorescence | 132 | 17 | 1.58 | 1.12 |

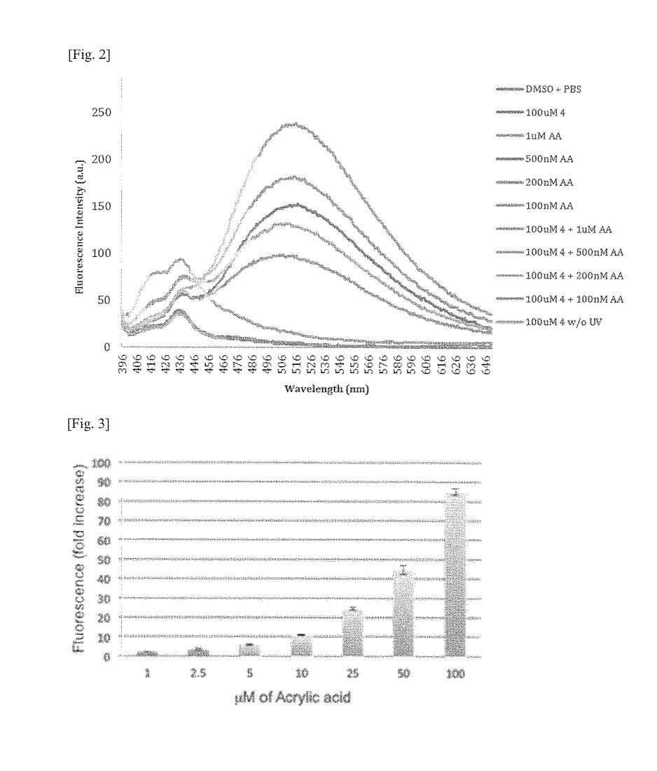

The emission spectra for each concentration of compound 4 and acrylic acid are plotted in FIG. 2. It can be observed that the concentration of acrylic acid or its derivatives thereof affects the fluorescent intensity. FIG. 3 also shows the relation between the fold increase in fluorescence upon the addition of acrylic acid at various concentrations to 100 μM of diaryltetrazole compound 4.

### Example 10—Reaction Monitoring of Photoactivated Cycloaddition Using HPLC and Fluorescence

10 mM of compound 4 was dissolved in dichloromethane and methanol (1:1). A separate solution of 10 mM of acrylic acid was dissolved in methanol. 10 μL of 10 mM of compound 4 and 10 μL of 10 mM of acrylic acid were then dissolved in 80 μL of methanol. After vigorous stirring, the mixture was irradiated with a 302 nm UV lamp for 0 seconds, 15 seconds, 30 seconds, 45 seconds, 60 seconds, 90 seconds and 120 seconds, respectively. An aliquot (10 μL) of reaction solution from each sample was withdrawn and immediately injected in the HPLC column. Compound 4 (denoted as A) and the pyrazoline product 4P (denoted as B) in each sample was monitored by UV absorbance at 254 nm and 370 nm. A linear gradient of 5 to 90% MeOH was applied after 3 minutes for 10 minutes, which is then kept constant for 10 minutes before a 7 minutes linear declining gradient of 90 to 5% MeOH. Compound 4 and product 4P were eluted at about 13.4 minutes and 12.4 minutes respectively.

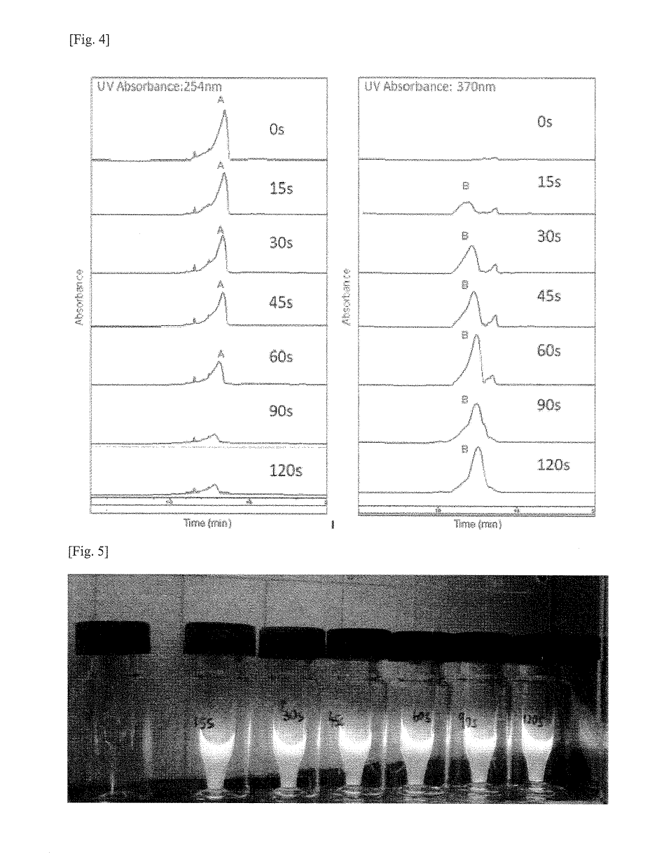

The kinetic reaction studies involving diaryltetrazole compound 4 and acrylic acid were carried out by monitoring with HPLC from 0 to 120 seconds as described above. Both the intermediate and product 4P were observed at 15 seconds. The reaction was observed to be completed within 90 seconds, and by this time, the starting material compound 4 was almost used up (see FIG. 4).

Fluorescence turn on of the reaction mixture occurred after 15 seconds. The left vial to the right vial are labelled as no UV activation, 15 seconds, 30 seconds, 45 seconds, 60 seconds, 90 seconds, 120 seconds of UV activation, respectively. All samples were prepared with the same method in which 1 μL of 1 mM of compound 4 and 1 μL of 1 mM of acrylic acid were dissolved in 98 μL methanol. Distances between all bottles of sample and UV lamp during activation were equal. The reaction mixture demonstrated a high turn on in fluorescence within 15 seconds (see FIG. 5).

### Example 11—Fluorescence Assay of Acrylic Acid Standards in LB Media and Minimum Media

Using the detection method of the present disclosure, fluorescence assay was carried out by contacting 100 μM of compound 4 with acrylic acid in LB media and minimum media for 1 min photoactivation. These media are commonly used for microbial synthesis of acrylic acid. Fluorescence was readily detected before the completion of photoactivation. This is significantly faster compared to the gas chromatography method exemplified in comparative example 1. This method also provides a higher throughput as compared to the HPLC method as illustrated in example 10 which only managed to complete elution of compound 4 and the pyrazoline product 4P by 13.4 minutes.

Accordingly, FIG. 15a shows the relationship between fluorescence intensity and the concentration of acrylic acid (labelled as AA) when the medium used is LB. FIG. 15b shows the relationship between fluorescence intensity and the concentration of acrylic acid (labelled as AA) when the medium used is a minimum medium. This example also demonstrates that the present method is capable of using the disclosed diaryltetrazole compounds for detecting acrylic acid in vitro.

### Example 12—pH Dependence of the Diaryltetrazole Reaction

To further study the reaction between compound 4 and acrylic acid, it was hypothesized that the large increase in fluorescence may be due to the benzoic acid group present in compound 4 and not the rest of the other exemplified compounds. Compound 4 contained the only ionisable group and thus, deprotonation may contribute to the high fluorescence emitted by pyrazoline product 4P which contains the carboxylic acid groups.

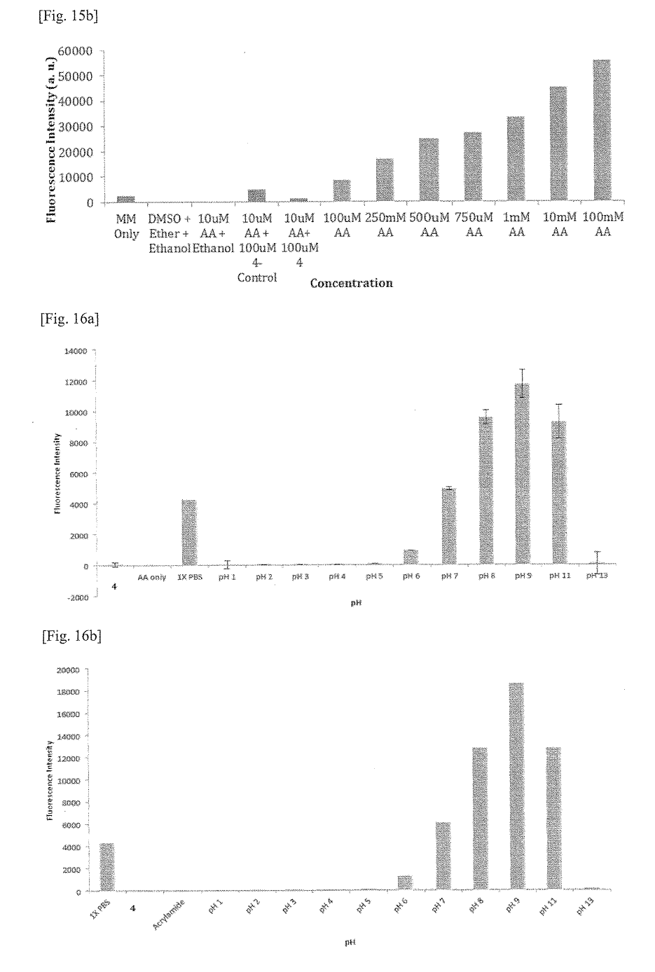

To substantiate the above, the reaction between compound 4 and acrylic acid (AA), and the reaction between compound 4 and acrylamide (a derivative), were carried out in buffers from pH 1 to pH 13. FIG. 16a and FIG. 16b showed that fluorescence of the pyrazoline product was stronger at basic pH with the highest fluorescence observed at a pH of around 8 to 11, particularly at pH 9. Deprotonation of the chromophore appears to result in higher fluorescence intensity of the pyrazoline product. The control experiments in these two figures showed that the diaryltetrazole probe was not fluorescent at all pH tested.

### Example 13—Detection of Acrylic Acid or its Derivatives Thereof

Experimental comparison between acrylic acid and one of its derivatives, acrylamide, has been carried out. Factors taken into consideration include, but not limited to, pH range, amount of acrylamide needed for detection and the impact of different oil media. Since compound 4 demonstrated the highest fluorescence based on the results of examples 1 to 7, this compound has been used for detection in this example.

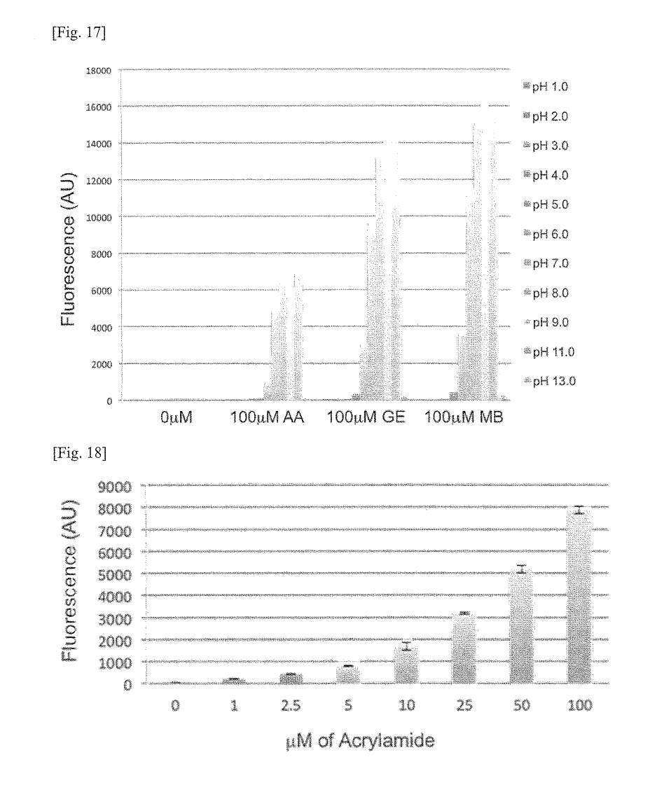

100 μM acrylic acid and 2 different grades of acrylamides, particularly gel electrophoresis acrylamide (GE) and molecular biology acrylamide (MB), were tested with 100 μM of compound 4 over the entire pH range. The reaction between compound 4 and acrylic acid/acrylamide was carried out in a phosphate buffer (having different pH as indicated) in 10% DMSO with a photoactivation time of 1 minute at 302 nm. Fluorescence was measured using a fluorescence microplate reader. Acrylic acid shows a signal detection peak at pH 11.0 whereas the acrylamide shows a peak at pH 9.0. The result of this comparison is shown in FIG. 17. Clearly, the detection method of the present disclosure is capable of detecting acrylate derivatives.

Compound 4 was also used to detect acrylamide in vitro. The reaction between compound 4 and acrylamide was carried out in phosphate buffer at pH 9.0 with 10% DMSO with a photoactivation time of 1 minute at 302 nm. Using 100 μM of compound 4, acrylamide concentrations from 1 μM to 100 μM were readily detectable with a fluorescence microplate reader as shown in FIG. 18.

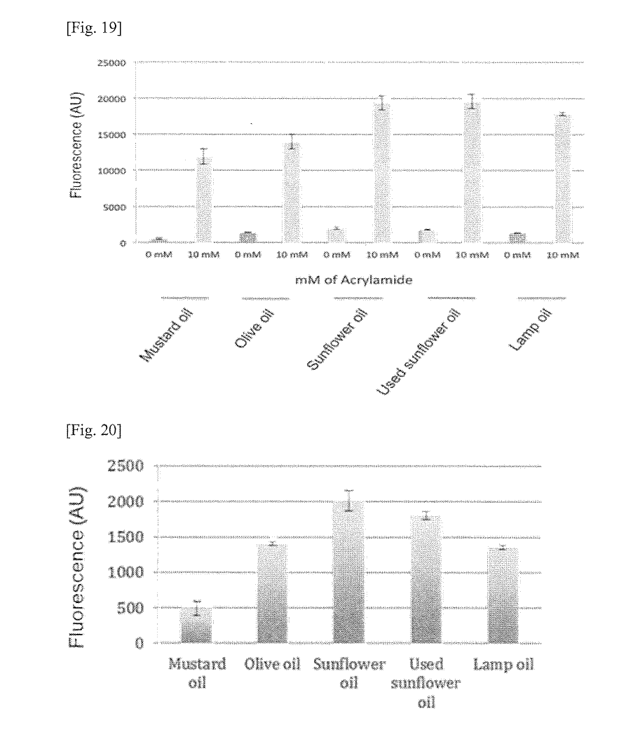

10% (v/v) of different oils in a phosphate buffer of pH 9.0 containing 1% Tween-20 were spiked in with 10 mM Acrylamide (gel electrophoresis grade). Controls were uncontaminated. Reactions of these mixtures were set up with 100 μM of compound 4 with a photoactivation time of 1 minute at 302 nm. Fluorescence was measured using a fluorescence microplate reader. Compound 4 is shown to be able to detect acrylamide in a complex organic/detergent mixture as supported by FIG. 19.

10% (v/v) of different oils in a phosphate buffer pH 9.0 containing 1% Tween-20 were tested with 100 μM compound 4 with a photoactivation time of 1 minute at 302 nm. Fluorescence was measured using a fluorescence microplate reader. The presence of a double bond in sunflower oil is likely to be the cause of this oil having the highest fluorescence measurement. Hence, the “double bond” may lead to inaccurate readings or may act as a contaminant when the present method is used. The results are shown in FIG. 20.

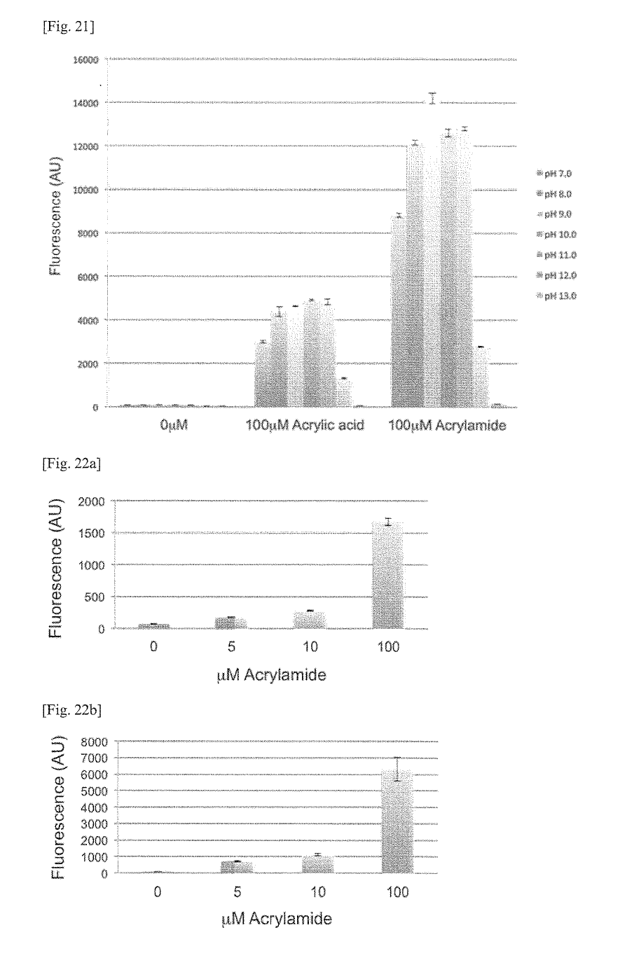

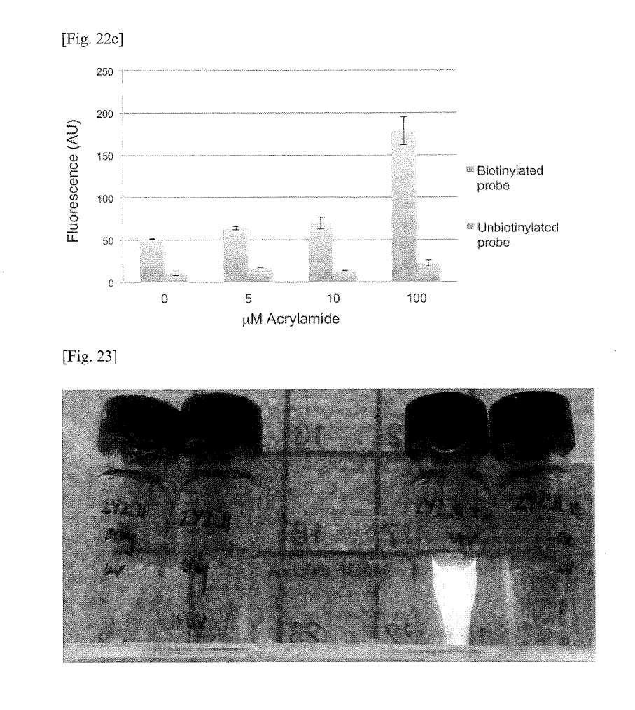

100 μM of acrylic acid and acrylamide (gel electrophoresis grade) were tested with 100 μM of compound 4 over a pH range of 7 to 13. The reaction between compound 4 and acrylic acid/acrylamide was carried out in a phosphate buffer (having different indicated pH) in 10% DMSO with a photoactivation time of 1 minute at 302 nm. Fluorescence was measured using a fluorescence microplate reader. Acrylic acid showed a signal detection peak at pH 10.0 whereas acrylamide had a fluorescent peak at pH 9.0. Results are shown in FIG. 21.

### Example 14—Detection Using Biotinylated Probe

Different concentrations of acrylamide in buffer pH 9.0 were tested with 100 μM biotinylated compound 4 (see FIG. 22a) or unconjugated compound 4 (see FIG. 22b) in solution with a photo activation time of 1 minute at 302 nm Fluorescence readings were taken using a fluorescence micro plate reader. Using streptavidin beads, biotinylated probe conjugated to acrylamide was pulled down and isolated (see FIG. 22c) and increased fluorescence was detected corresponding to increasing amounts of acrylamide. Controls included unbiotinylated probe for pull down that showed background bead fluorescence.

Based on this, biotinylation improves the speed and accuracy of the present detection method.

From FIG. 23, it can be observed that the biotinylated probe also works on acrylic acid. The two vials on the left contains only biotinylated compound 4. The first (leftmost) vial on the left was exposed to UV for 2 minutes but showed no fluorescence. The second vial on the left was not exposed to UV. On the other hand, the two vials arranged on the right contains biotinylated compound 4 mixed with acrylic acid. Fluorescence was observed within 2 minute of photoactivation via UV for the first (left) vial arranged on the right. The second (rightmost) vial arranged on the right did not reveal any fluorescence as it was not exposed to UV. In this set of experiment as shown in FIG. 23, the probe contained compound 4 conjugated to a biotin group.

### Example 15—In Vivo Detection

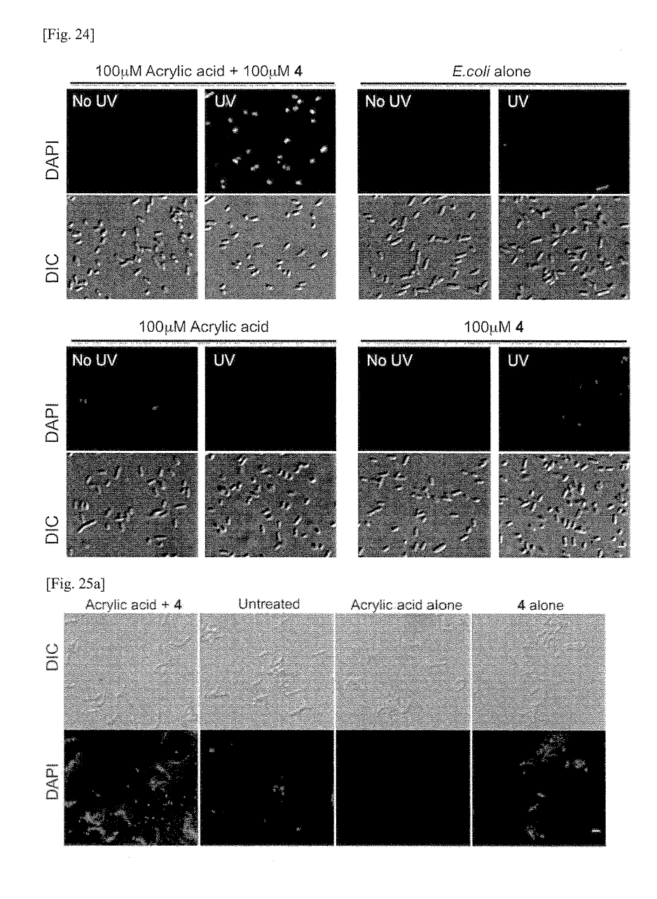

The use of compound 4 as an acrylic acid sensor in vivo was tested in this example. E. coli cells were grown to late log phase (OD₆₀₀˜1.0) and treated with 100 μM acrylic acid for 10 minutes at 37° C. Upon washing, cells were treated with 100 μM of compound 4 and incubated at 37° C. for 30 min in the dark. The cells were washed, pelleted, suspended in 1×PBS and mounted on a slide. They were then exposed to UV light at 302 nm for 1 minute and imaged under a fluorescence microscope (using DAPI filters) after about 2 hours of recovery at room temperature. Control cells include untreated cells; cells treated with either acrylic acid or compound 4 alone and without UV treatment. The results in FIG. 24 showed that the bacterial cells were only fluorescent in the presence of both acrylic acid and compound 4.

Acrylic acid has been shown to be produced as a metabolic inteimediate in two bacterial species such as, but not limited to, Clostridium propionicum and Megasphaera elsdenii. In these microbes, the reduction of lactic acid to propionic acid proceeds via an acrylyl-CoA intermediate.

To test the diaryltetrazole sensor for in vivo production of acrylic acid, we applied compound 4 to C. propionicum cells. C. propionicum cells were grown to late log phase (OD₆₀₀˜1.0) in an anoxia chamber and treated with 100 μM of compound 4. After incubating at 37° C. for 30 min in the dark, cells were washed, pelleted, suspended in PBS buffer and mounted on a slide. Cells were exposed to 302 nm UV light for 1 minute and imaged under a fluorescence microscope (using DAPI filters) after about 2 hours of recovery at room temperature. Control cells include cells treated with acrylic acid and compound 4 to observe positive fluorescence; untreated cells and cells treated with acrylic acid alone and without UV treatment. The results in FIG. 25a and FIG. 25b showed that cells were fluorescent in both the control experiment where 100 μM of acrylic acid was added and in C. propionicum cells. FIG. 25b shows the fluorescence results of the C. propionicum lysates. This indicates the production of acrylic acid intermediates in these cells and the diaryltetrazole probe is capable of detecting them.

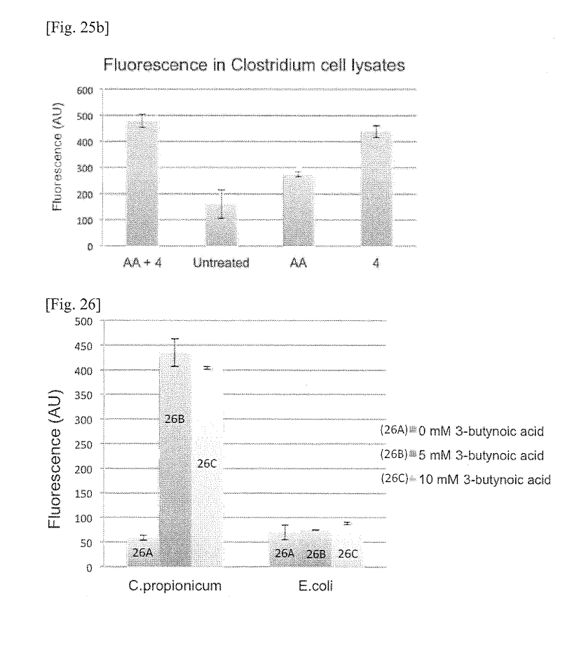

FIG. 26 shows the fluorescence signal of bacterial cell lysates from C. propionicum and E. coli grown in media either not treated (26a) or treated with 5 and 10 mM 3-butynoic acid (26b and 26c respectively). The cell lysates were treated with 500 mM of diaryltetrazole compound 4 for fluorescence detection. These cell lysates from the same experiment as demonstrated in the above paragraph were used to quantitatively measure the fluorescence signal using a fluorescence plate reader. Hence, this shows that the diaryltetrazole probe of the present disclosure can be used to detect acrylic acid or its derivatives thereof. 3-Butynoic acid, an acyl CoA dehydrogenase inhibitor, may be capable of promoting accumulation of acrylyl CoA in cells. In C. propionicum, acrylyl CoA is normally converted to propionyl CoA. However, in the presence of 3-butynoic acid, this reaction is inhibited. Thus, in cells containing 3-butynoic acid, the fluorescence signal is higher, indicating a higher acrylic acid content. C. propionicum naturally produces acrylic acid but E. Coli. may not naturally produce acrylic acid.

### Comparative Example 1—Gas Chromatography

As described above, compound 4 was used in further experiments to detect acrylic acid. The reaction between compound 4 and acrylic acid was carried out in water with a photoactivation time of 1 minute at 302 nm. Using 100 μM of 4, acrylic acid concentrations from 1 μM to 100 μM were readily detectable with a fluorescence microplate reader (see FIG. 3 and example 9). This result was compared with GC detection of acrylic acid without using the diaryltetrazole compounds envisaged by the present disclosure.

Samples for GC analysis have to be extracted into a volatile organic solvent such as ether, before they can be analyzed. The detection limit for GC analysis is 250 μM (see table 3 below) while 100 μM of the extracted acrylic acid was readily detectable using the fluorescence assay disclosed in examples 9 and 11. The time consuming process of sample extraction for GC does not provide a method of high throughput screening.

TABLE 3

| Concentration of acrylic acid | | Peak Intensity |
|---|---|---|
| 100 | mM | 22,000,000 |
| 10 | mM | 22,000,000 |
| 1 | mM | 5,700,000 |
| 750 | μM | 2,600,000 |
| 500 | μM | 2,600,000 |
| 250 | μM | 1,350,000 |
| 100 | μM | 650,000 |
| 10 | μM | 350,000 |
| 0 | μM | 700,000 |

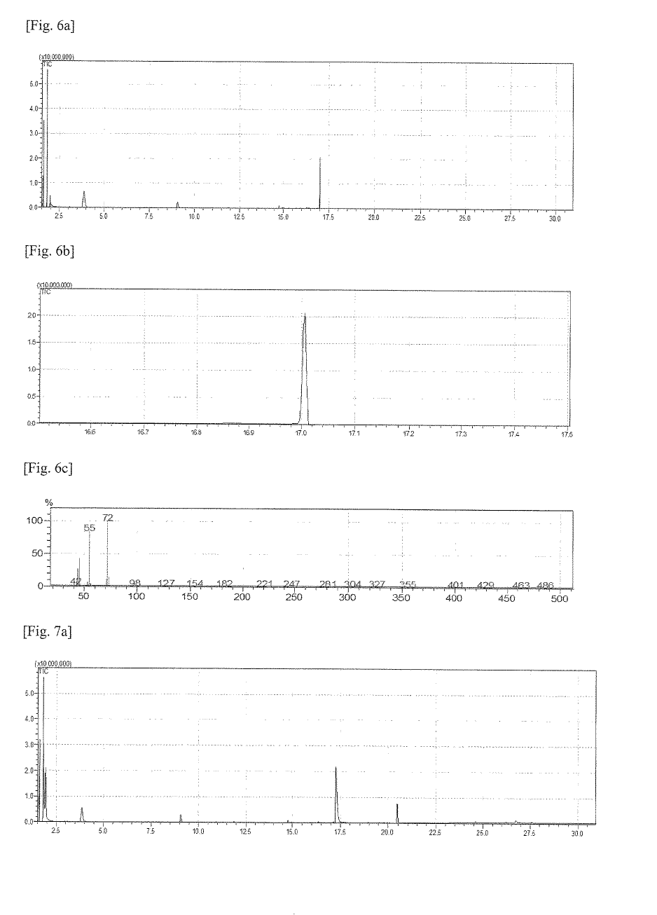

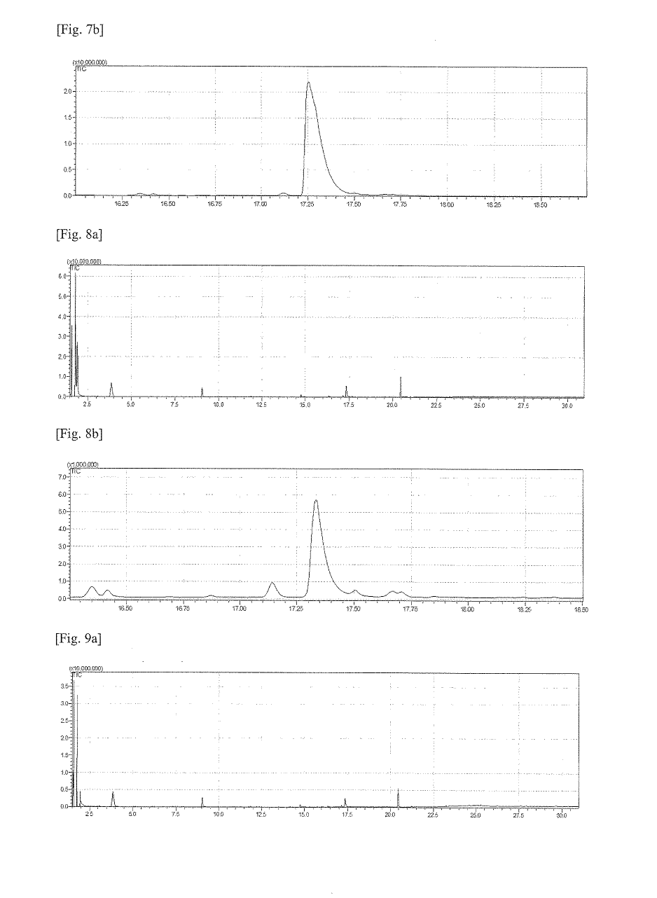

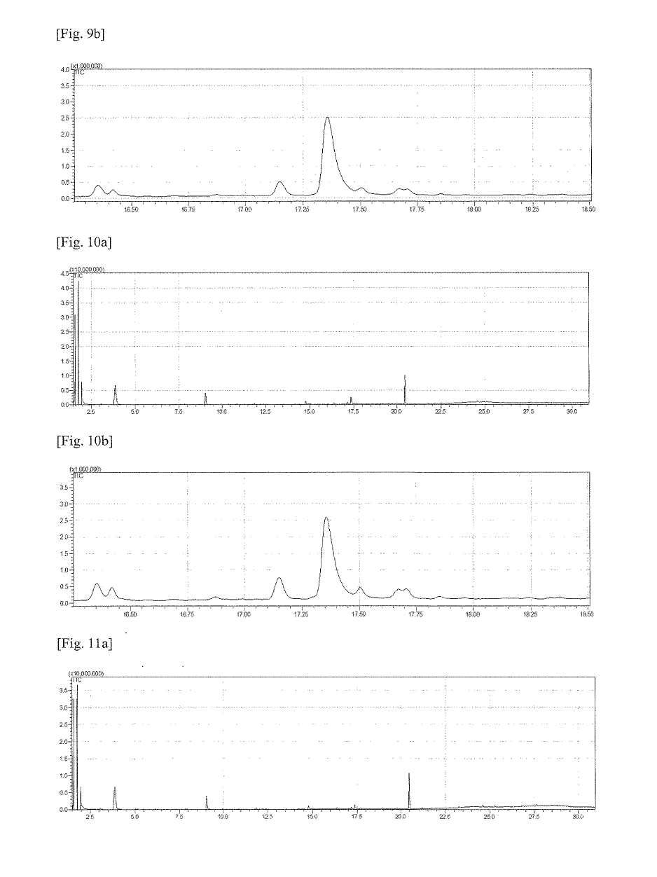

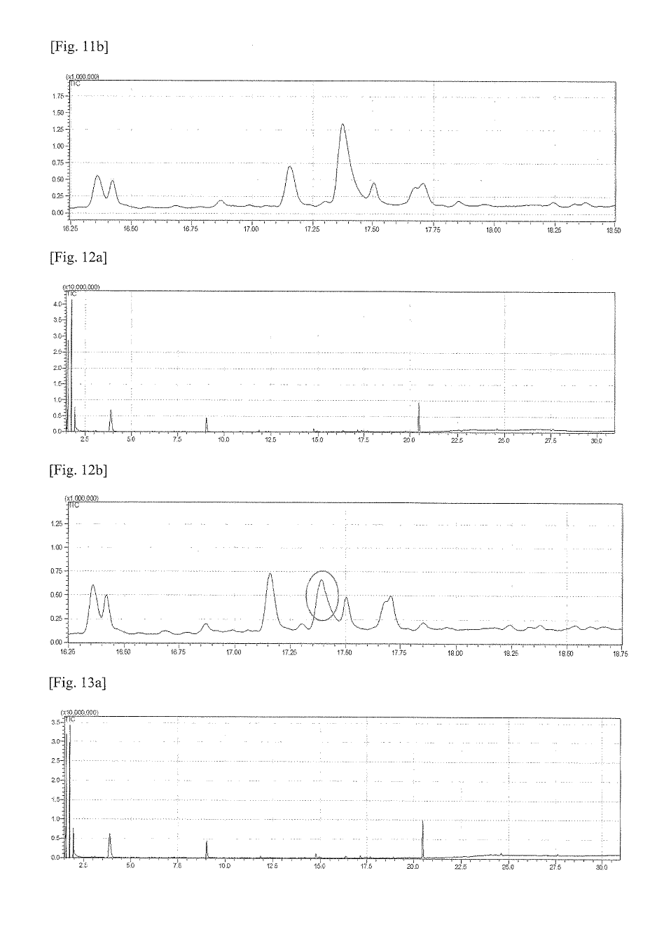

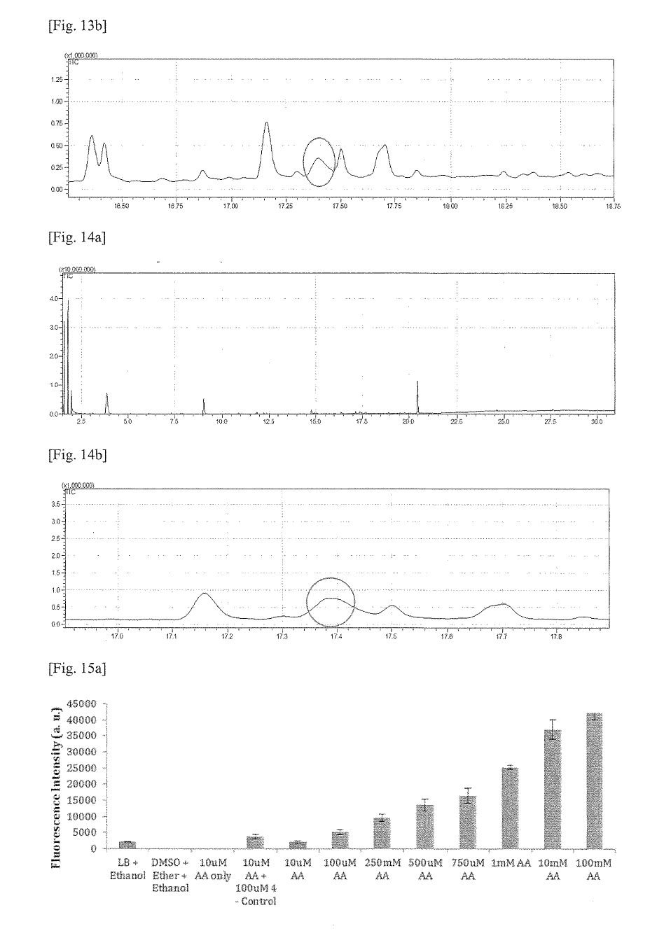

Acrylic acid (6.8 μL, 99 μmol) was dissolved in Lysogeny broth (LB) medium (993.2 μL) and stirred vigorously for 10 seconds before being diluted to their respective concentration with LB. A Minimum medium may be used to replace the LB medium. 1000 μL of samples of each concentration were transfer to a 2 mL eppendorf tube and acidified with 30 to 50 μL of 5M HCl. Each sample was stirred vigorously for about 10 seconds and left to stand for 3 to 5 minutes. pH of each sample were tested to ensure pH≦2. Ether (1000 μL×2) was then added to the acidified samples for extraction. Combined ether layers were then concentrated to about 60 μL for gas chromatography mass spectrometry. Extracted samples were not allowed to evaporate completely as it will affect the GCMS result. Acrylic acid was detected at a retention time of around 17.4 minutes. The GCMS results for various concentrations of acrylic acid are shown in FIG. 6a to FIG. 14c. FIG. 6c shows the mass spectrometry data of the acrylic acid peak when the concentration of acrylic acid is at 100 mM. This GC method is slower compared to the method of the present disclosure which detected acrylic acid before 90 seconds or even before 1 minute.

### INDUSTRIAL APPLICABILITY

The method as defined herein enables the detection of the presence or absence of acrylic acid or its derivatives thereof by contacting or mixing a diaryltetrazole as described above with a sample containing the acrylic acid or its derivatives thereof. Photoactivation of such a mixture may cause the mixture to fluorescent if acrylic acid or its derivatives are detected. Advantageously, this fluorimetric sensing method may provide a method of rapidly detecting acrylic acid or its derivatives thereof without the need for tedious sample preparation such as those of GCMS and HPLC. Chemical derivatization and bulky detection apparatus may be eliminated since the detection relies on fluorescence.

Further advantageously, the present method may also allow high throughout detection compared to conventional methods such as GCMS, liquid chromatography or HPLC etc.

The present method may also utilize non-cytotoxic compounds as a detection probe. Hence, the present method and probe may be used to detect acrylic acid or its derivatives thereof in vitro and in vivo.

Accordingly, the probe as described herein may be used in the present detection method as described above and such a probe may possess the above advantages. The probe may be further biotinylated to enhance detection efficiency and accuracy. A kit comprising such a probe when used or used for detecting acrylic acid or its derivatives thereof may also possess the above advantages.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A method for detecting the presence or absence of acrylic acid or its derivatives thereof in a sample, the method comprising:
(a) introducing a probe comprising a diaryltetrazole compound to the sample;
(b) exposing said sample to light; and
(c) detecting the presence or absence of acrylic acid or its derivatives thereof in the sample based on fluorescence emitted by the sample after said operation (c).

2. The method according to claim 1, wherein the diaryltetrazole compound has the formula (I): 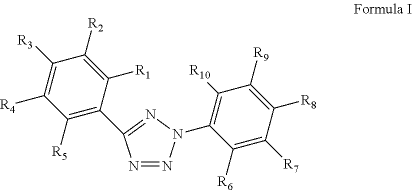
wherein each of R1 to R10 is independently selected from the group consisting of hydrogen, oxygen, sulfur, halogen, hydroxyl, optionally substituted alkyl, optionally substituted acyl, optionally substituted ester, optionally substituted amino, optionally substituted amine, optionally substituted amide, optionally substituted carboxylic acid, optionally substituted carbonyl, optionally substituted urea, optionally substituted alkoxy, optionally substituted alkyloxy, optionally substituted alkenyl, optionally substituted alkynyl, optionally substituted sulfonamide, optionally substituted aminosulfonamide, optionally substituted sulfonylurea, optionally substituted oxime, optionally substituted cycloalkyl, optionally substituted aryl, optionally substituted heterocycloalkyl and optionally substituted heteroaryl.

3. The method according to claim 2, wherein said diaryltetrazole compound is selected from the group consisting of:

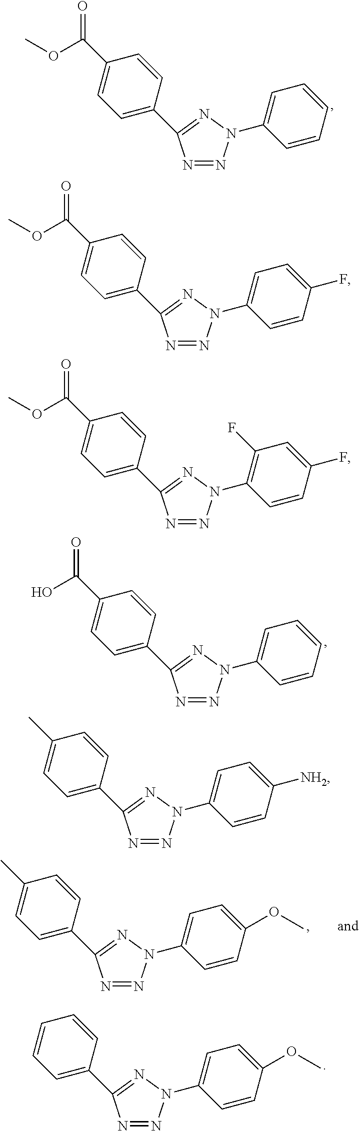

4. (canceled)

5. The method according to claim 1, wherein said probe is biotinylated.

6. The method according to claim 1, wherein said exposure occurs at a wavelength in the range of 10 nm to 1 mm.

7. The method according to claim 6, wherein said wavelength is 302 nm.

8. The method according to claim 1, wherein the operation of exposure further comprises the operation of forming a reactive intermediate.

9. The method according to claim 8, wherein said reactive intermediate is a compound comprising a nitrile imine dipole.

10. The method according to claim 1, wherein said fluorescent sample after exposure to light is a cycloadduct comprising a fluorescent pyrazoline.

11. The method according to claim 1, wherein said acrylic acid or its derivatives thereof is present at a concentration of at least 100 nM before introducing the probe to said sample.

12. The method according to claim 1, wherein the diaryltetrazole compound introduced is present at a concentration of at least 1 nM in said sample.

13. The method according to claim 1, wherein the exposure of said sample to light occurs under alkaline conditions.

14. The method according to claim 1, wherein said sample is a microorganism.

15. The method according to claim 14, wherein said microorganism is a bacterium.

16. A probe for detecting the presence or absence of acrylic acid or its derivatives thereof in a sample, wherein the probe comprises a diaryltetrazole compound.

17. The probe according to claim 16, wherein the diaryltetrazole compound has the formula (I): 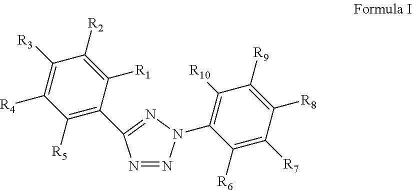
wherein each of R1 to R10 is independently selected from the group consisting of hydrogen, oxygen, sulfur, halogen, optionally substituted alkyl, optionally substituted acyl, optionally substituted ester, optionally substituted amino, optionally substituted amine, optionally substituted amide, optionally substituted carboxylic acid, optionally substituted carbonyl, optionally substituted urea, optionally substituted alkoxy, optionally substituted alkyloxy, optionally substituted alkenyl, optionally substituted alkynyl, optionally substituted sulfonamide, optionally substituted aminosulfonamide, optionally substituted sulfonylurea, optionally substituted oxime, optionally substituted cycloalkyl, optionally substituted aryl, optionally substituted heterocycloalkyl and optionally substituted heteroaryl.

18. The probe according to claim 17, wherein said diaryltetrazole compound is selected from the group consisting of:

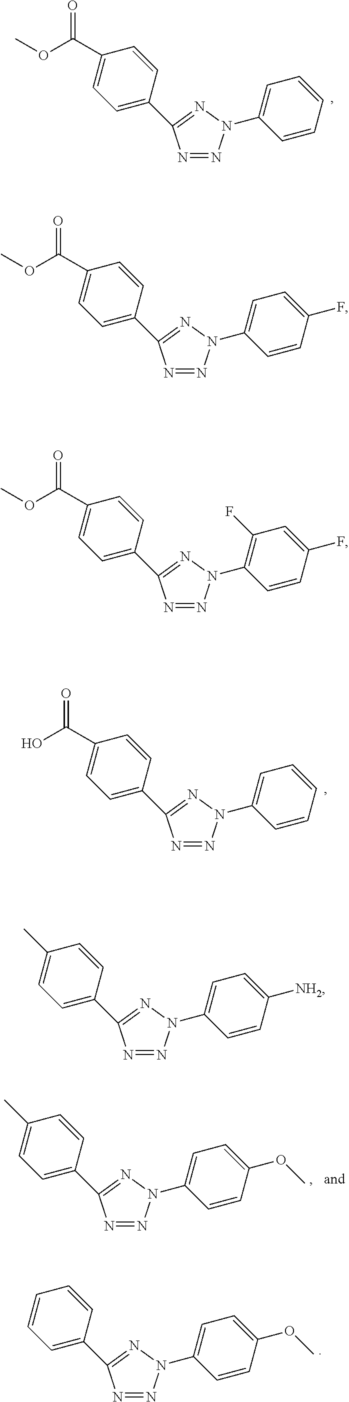

19. (canceled)

20. The probe according to claim 16, wherein said probe is biotinylated.

21. Use of a probe for detecting the presence or absence of acrylic acid or its derivatives thereof in a sample, wherein the probe comprises a diaryltetrazole compound.

22. The probe according to claim 16 for detecting the presence or absence of acrylic acid or its derivatives, wherein the probe is contacted with the acrylic acid or its derivatives.
